(19) United States
(12) Patent Application Publication
(10) Pub. No.: US 2025/0262351 A1
(43) Pub. Date: Aug. 21, 2025

(54) NORBORNENE-MODIFIED DECELLULARIZED EXTRACELLULAR MATRICES AND METHODS OF MAKING AND USING THE SAME

(71) Applicant: The Trustees of Indiana University, Bloomington, IN (US)

(72) Inventors: Chien-Chi Lin, Indianapolis, IN (US); Thuy Van Duong, Bloomington, IN (US)

(21) Appl. No.: 19/053,679

(22) Filed: Feb. 14, 2025

Related U.S. Application Data
(60) Provisional application No. 63/554,603, filed on Feb. 16, 2024.

(51) Int. Cl. A61L 27/36; A61L 27/52; B33Y 70/00; C08L 101/02; C12N 5/00

(52) U.S. Cl. CPC A61L 27/3633; A61L 27/52; B33Y 70/00; C08L 101/02; C12N 5/0012; A61L 2430/34; A61L 2430/40; C08L 2312/06

(57) ABSTRACT

A composition comprising decellularized extracellular matrices, hydrogels comprising the same, and methods of making the same are disclosed.

### CROSS-REFERENCE TO RELATED APPLICATION

This application claims priority to U.S. Application Ser. No. 63/554,603, filed Feb. 16, 2024, which is incorporated by reference herein in its entirety.

### STATEMENT ON FEDERALLY SPONSORED RESEARCH OR DEVELOPMENT

This invention was made with government support under DK127436 and CA227737 awarded by the National Institutes of Health. The Government has certain rights in the invention.

### SEQUENCE LISTING

This application also incorporates by reference the material in the ST.26 XML file titled SequenceListing.xml which was created on Jan. 23, 2024 and is 1,725 bytes.

### FIELD

Embodiments disclosed herein are directed to hydrogels, and more particularly to hydrogels comprising norbornene-modified decellularized extracellular matrices (dECM).

### BACKGROUND

Decellularized extracellular matrices (dECM), a fibrillar network of cell-free proteins and glycosaminoglycans (GAG), have been increasingly used to construct bioactive scaffolds for promoting tissue regeneration. All dECM contain abundant bioactive ligands and micro/nanofiber structures, which benefit cell attachment and morphogenesis. For example, decellularized small intestine submucosa (dSIS) has a long history in biomedical applications and has been approved by the US Food and Drug Administration (FDA) for clinical applications including wound dressing, ureteral reconstruction, and hernia repair. The highly bioactive and fibrillar dSIS has proven advantageous in facilitating cell attachment, migration, and differentiation. Furthermore, dSIS exhibits high viscoelasticity and has been used to establish in vitro cancer models and for screening of anti-cancer therapeutics.

Even with its inherent bioactivity, the use of dECM is not without limitations. Similar to other protein/GAG-based matrices, almost all dECM hydrogels for regenerative medicine applications are physically crosslinked. Three notable limitations are the lack of adaptable matrix mechanics, long gelation time, and poor stability for long-term cell culture. In this regard, chemically crosslinked dECM hydrogels offer an attractive alternative to the physically gelled matrices owing to the preservation of bioactive cues and tailorable biophysical properties. For example, Elomaa et al. recently reported the modification of dSIS with methacryloyl groups, creating dSIS-MA amenable for light and radical-initiated photocrosslinking and 3D bioprinting. In another recent example, Hewawasam et al. modified commercially available human dECM with sulfhydryl groups (i.e., dECM-SH), enabling its crosslinking with methacrylated polymer via Michael-type addition. When the a-methacrylates were in excess, the Michael-type dECM hydrogels could be dynamically stiffened via secondary free-radical mediated chain-polymerization of methacrylate groups. Nonetheless, the crosslinking of methacrylate containing dECM hydrogels via chain-growth polymerizations generated high concentrations of propagating radicals with potential cytotoxicity. Another challenge in using chain-polymerized dSIS-MA hydrogels was the difficulty in decoupling matrix stiffness from the contents of bioactive motifs.

The highly efficient and cytocompatible step-growth photopolymerization based on thiol-norbornene photo-click chemistry offers highly tunable and modular controls of hydrogel properties, and can be readily immobilized with bioactive molecules during hydrogel crosslinking. The rapid and modular step-growth thiol-norbornene photopolymerization has been widely used for in situ cell encapsulation and increasingly adapted for biofabrication and bioprinting applications, including digital light processing (DLP) bioprinting, volumetric bioprinting, and extrusion-based bioprinting. For example, Kim et al. synthesized methylcellulose-norbornene (MCNB) and used it as a highly viscous thermo- and photo-responsive bioink. Through using an extrusion-based bioprinter, MCNB and gelatin-norbornene (GelNB) were printed together to produce various 3D structures. The same group also optimized low-viscosity poly(ethylene glycol)-norbornene (PEGNB) as a resin for thiol-norbornene-based DLP bioprinting. Later, GelNB was adapted in a similar fashion for DLP bioprinting to create structurally complex thiol-norbornene hydrogels. Uniquely, the printed GelNB hydrogels were amenable to post-printing conjugation of additional bioactive peptides, such as a vascular endothelial growth factor (VEGF)-mimetic peptide QK, rending the bioprinted GelNB hydrogels conductive to vascularization. In another example, Rizzo et al. explored GelNB in 3D volumetric printing to create complex and low-defect 3D objects within seconds, overcoming the sometimes time-consuming layer-by-layer additive manufacturing. Nonetheless, no dECM (including dSIS) has been modified with norbornene groups for step-growth photopolymerization and biofabrication applications.

### SUMMARY

Therefore, a need remains for development of dECM (including dSIS) modified with norbornene groups for step-growth photopolymerization and biofabrication applications. The present disclosure addresses these needs.

The present disclosure provides norbornene-derivatized decellularized extracellular matrices (dECM-NB) synthesized through reacting the primary amines on dECM with carbic anhydride, with triethylamine (TEA) as a base catalyst. The dECM-NB was readily crosslinked with poly(ethylene glycol)-tetra-thiol (PEG4SH) to form thiol-norbornene hydrogels. To demonstrate the potential of using dECM-NB hydrogels as an angiogenic matrix, functional vascular network formation through in situ encapsulation of human umbilical vein endothelial cells (HUVECs), either as single cell suspension or as pre-formed spheroids, was assessed. Moreover, the suitability of using dECM-NB hydrogels for in vitro pancreatic cancer cell culture was evaluated. Granular hydrogels were further developed by annealing individual dECM-NB microgels. Finally, dECM-NB was used as a bioink for extrusion-based and DLP bioprinting.

According to an embodiment, In an aspect, either alone or in combination with any other aspect, a composition comprises decellularized extracellular matrices, wherein the decellularized extracellular matrices comprise one or more norbornene substituents.

In an embodiment, either alone or in combination with any other embodiment, the one or more norbornene substituent is attached to one or more primary amines of the decellularized extracellular matrices.

In an embodiment, either alone or in combination with any other embodiment, the decellularized extracellular matrices are derived from a mammal.

In an embodiment, either alone or in combination with any other embodiment, a macromer for producing a hydrogel comprises decellularized extracellular matrices, wherein the decellularized extracellular matrices comprise one or more norbornene substituents.

In an embodiment, either alone or in combination with any other embodiment, a method for making a photocrosslinkable macromer comprises reacting decellularized extracellular matrices (dECM) with carbic anhydride and a nucleophilic catalyst to yield a norbornene-functionalized dECM.

In an embodiment, either alone or in combination with any other embodiment, the dECM comprises one or more primary amines.

In an embodiment, either alone or in combination with any other embodiment, the photocrosslinkable macromer comprises one or more amide linkages formed by the one or amines of the dECM and a carboxyl domain of the norbornene.

In an embodiment, either alone or in combination with any other embodiment, the nucleophilic catalyst comprises triethylamine.

In an embodiment, either alone or in combination with any other embodiment, a method for making a hydrogel comprises reacting decellularized extracellular matrices (dECM) with carbic anhydride and a nucleophilic catalyst to yield a norbornene-functionalized dECM; and reacting the norbornene-functionalized dECM with at least one thiol-bearing compound.

In an embodiment, either alone or in combination with any other embodiment, the reacting the norbornene-functionalized dECM with at least one thiol-bearing compound comprises exposing the norbornene-functionalized dECM and the thiol-bearing compound to electromagnetic radiation having a wavelength from 365 nm to 405 nm.

In an embodiment, either alone or in combination with any other embodiment, the thiol-bearing compound is selected from the group consisting of 1,4-dithiothreitol, 4-arm thiolated PEG, a peptide that includes more than one cysteine residue, a natural or thiolated protein, thiolated gelatin, thiolated hyaluronic acid, thiolated collagens, and combinations thereof.

In an embodiment, either alone or in combination with any other embodiment, the thiol-bearing compound comprises dithiothreitol.

In an embodiment, either alone or in combination with any other embodiment, the hydrogel comprises an orthogonal thiol-norbornene hydrogel.

In an embodiment, either alone or in combination with any other embodiment, a hydrogel is produced according to any method disclosed herein, including any combination of described steps to arrive at the hydrogel.

In an embodiment, either alone or in combination with any other embodiment, the hydrogel is used for a purpose selected from the group consisting of as a substrate for short-term 2D cell culturing, 3D in situ encapsulation of viable cells, controlled delivery of drugs and other bioactive materials, tissue engineering, tissue regeneration, creating micropores in a bulk polyethylene glycol-based hydrogel, creating a sacrificial material in a 3D bioprinted article, creating a sacrificial material in a photolithography-based biofabrication product, and creating a sacrificial bioink for extrusion-based or photolithography-based 3D bioproducts.

These and other embodiments, forms, features, and aspects of the disclosure will become more apparent through reference to the following description, the accompanying figures, and the claims. This summary is not intended to identify key or essential features of the claimed subject matter, nor is it intended to be used as an aid in limiting the scope of the claimed subject matter. Additionally, it is to be understood that the features of the various embodiments described herein are not mutually exclusive and can exist in various combinations and permutations. Furthermore, it is envisioned that alternative embodiments may combine features of two or more of the above-summarized embodiments. Further embodiments, forms, features, and aspects of the present application shall become apparent from the description and figures provided herewith.

### BRIEF DESCRIPTION OF THE DRAWINGS

The concepts described herein are illustrative by way of example and not by way of limitation in the accompanying figures. For simplicity and clarity of illustration, elements illustrated in the figures are not necessarily drawn to scale. Where considered appropriate, references labels have been repeated among the figures to indicate corresponding or analogous elements.

FIG. 1 shows fresh bovine small intestine submucosa tissues after removal of mesenteric tissue, mucosal epithelium, and lamina propria decellularized using sodium dodecyl sulphate and Triton-X. The SIS tissues transformed into smooth, white strips. Following the decellularization process, lyophilization, and blending, the dSIS tissues transformed into a white powder and then, gradually dissolved in an acidic enzymatic solution comprising HCl and pepsin. Fluoraldehyde assay was employed to quantify the amine content on the dSIS backbones. Those primary amines served as nucleophiles for reaction with carbic anhydride to form amide-linked norbornene dSIS (e.g., dSIS-NB). Following modification and dialysis, dSIS-NB appeared in a sponge-like form. dSIS-NB sponge was subsequently reconstituted in PBS for future use.

FIG. 2 shows the modification of dSIS with carbic anhydride, as confirmed by ¹H NMR spectroscopy.

FIG. 3 shows in situ photo-rheometry showing the rapid gelation kinetics of thiol-norbornene cross-linking between 0.8 wt % dSIS-NB, 0.8 wt % PEG4SH, and 5 mM LAP under 405 nm light 28.8 mW/cm² light intensity.

FIG. 4 shows a schematic of thiol-norbornene photocrosslinking to form dSIS-NB hydrogel. In the presence of a water-soluble photoinitiator (e.g., lithium phenyl-2,4,6-trimethylbenzoylphosphinate or LAP), this gelation could be initiated under 365 nm or 405 nm light exposure.

FIG. 5 shows various hydrogel shapes, such as star and fiber fabricated using the same formulation with the in situ photo-rheometry under 365 nm light 2.9 mW/cm² light intensity.

FIG. 6 shows tunable stiffness of thiol-ene dSIS-NB hydrogels.

FIG. 7 shows G′ vs time for various concentrations of PEG4SH.

FIG. 8 shows top view area change of 0.8 wt % dSIS-NB hydrogels with different concentration of PEG4SH after 24 h.

FIG. 9 shows scanning electron microscope images of the dSIS and thiol-norborene dSIS-NB hydrogels with different PEG4SH concentrations.

FIG. 10 shows microspheroids, which contained either red fluorescent protein-labeled pancreatic cancer cells (COLO-357), green fluorescent protein-labeled immortalized human pancreatic cancer-associated fibroblasts (CAFs), or a combination of both, encapsulated within 0.8 wt % dSIS-NB crosslined by with 0.4 wt % PEG4SH with 5 mM LAP.

FIG. 11 shows COLO-357-CAF microspheroids within 0.8 wt % dSIS and 0.4 wt % Type I collagen for 3 days.

FIG. 12 shows the longest distance of COLO-357s from the center of the spheroids quantified using 3D confocal images of COLO-357-CAF spheroids.

FIG. 13 shows stress relaxation of 0.8 wt % dSIS-NB hydrogel crosslinked by different concentrations of PEG4SH (5 mM LAP, 365 nm light, 2 min exposure, 2.9 mW/cm² light intensity).

FIG. 14 shows morphology of COLO-357s and CAFs within dSIS-NB hydrogel.

FIG. 15 shows the aspect ratio (length/width) of COLO-357 over 3 days.

FIG. 16 shows HUVEC microspheroids within both native dSIS and thiol-norbornene dSIS-NB hydrogels with different stiffnesses (e.g., different PEG4SH concentrations) for 3 days. Cellular protrusion/sprouting was not visible in the first two 24 hours post-encapsulation. In contrast, significant protrusion along the periphery of the HUVEC spheroids was notable in all three dSIS-NB hydrogels. Even 2 days later, the number and length of these initial sprouts within the dSIS hydrogel exhibited only trivial growth compared to those in the dSIS-NB hydrogels. The sprouting lengths were considerably longer within dSIS-NB gels compared to the dSIS gels.

FIG. 17 shows 3D confocal images of HUVEC spheroids and their sprouts within the dSIS and dSIS-NB hydrogels on day 3.

FIG. 18 shows the lengths of the sprouts.

FIG. 19 shows stiffness of native dSIS gels.

FIG. 20 shows immunostained images of HUVEC spheroids with integrin beta1 (ITB1) and F-actin within dSIS and dSIS-NB hydrogels after 24 h of encapsulation.

FIG. 21 shows a cross-sectional view derived from a 3D confocal image on day 3 of a HUVEC spheroid within the 0.8 wt % dSIS-NB hydrogel crosslinked by 0.4 wt % PEG4SH (green is F-actin, blue is nucleus).

FIG. 22 shows HUVEC spheroids aligned as a Y-shape within 0.8 wt % dSIS-NB hydrogel crosslinked by 0.4 wt % PEG4SH with 5 mM LAP under exposure of 365 nm light for 2 min.

FIG. 23 shows the luminal structure of the micro neo-vasculature sprouting from the HUVEC spheroid within the 0.8 wt % dSIS-NB hydrogel containing 0.4 wt % PEG4SH on day 3.

FIG. 24 shows 3D confocal live/dead images of encapsulated HUVECs within dSIS and thiol-norbornene dSIS-NB hydrogels over 7 days. HUVECs were suspended in 0.8 wt % dSIS or dSIS-NB hydrogels containing varying concentrations of PEG4SH, along with 5 mM LAP. Then, the dSIS-NB precursor was exposed to 365 nm light (2.9 mW/cm²) for 2 minutes and the dSIS solution was physically crosslinked at 37° C. for 30 min. At a similar cell density of about 5×10⁶ cells/mL for encapsulation, the HUVECs in the dSIS-NB gels exhibited rapid proliferation, interconnection, and gel contraction.

FIG. 25 shows HUVEC viability within dSIS and dSIS-NB hydrogels on Day 0.

FIG. 26 shows the coverage of HUVECs on 2D confocal slices over 7 days, reflecting the vascular network formation within the hydrogels.

FIG. 27 shows size changing of encapsulated HUVEC gels over 7 days.

FIG. 28 shows stiffness changing of encapsulated HUVEC gels over 7 days.

FIG. 29 shows dextran occupied the lumen structure of microvessels once immersing a HUVEC-encapsulated 0.8 wt % dSIS-NB hydrogel crosslinked by 0.8 wt % PEG4SH hydrogel into 40 kDa FITC-Dextran on day 5 for 16 h.

FIG. 30 shows fluorescent microparticles perfused into a microvascular network which formed inside a u-slide for 3 days.

FIG. 31 shows a 3D confocal cross-sectional view of the microvascular network with trapped fluorescent microparticles in its lumen.

FIG. 32 shows microgels that will be used to form microporous annealed particle (MAP) hydrogels. The microgels were collected, rinsed with PBS 5 times, and observed under a bright-field microscope. The average diameter of the 228 microgels was measured to be 211.8 μm, with a coefficient of variation (c.v.) of 38.27.

FIG. 33 shows microgels annealed using 4-arm PEG-tetrazine (PEG4Tz) though inverse electron demand Diels-Alder click chemistry to form microporous annealed particle hydrogels.

FIG. 34 shows a MAP hydrogel with 8-mm diameter and 1-mm thickness.

FIG. 35 shows 3D confocal images showing FITC-Dextran penetrated the empty spaces within the MAP hydrogel.

FIG. 36 shows HUVEC microspheroids encapsulated within both 2 wt % dSIS-NB bulk hydrogel and MAP hydrogel over 3 days.

FIG. 37 shows viability of HUVEC spheroids within 2 wt % dSIS-NB bulk hydrogels and MAP hydrogel.

FIG. 38 shows a histogram of microgel diameter (n=228, mean=211.8 μm, coefficient variation=38.27).

FIG. 39 shows a schematic description of extrusion-based bioprinting.

FIG. 40 shows a schematic of digital light processing (DLP) bioprinting.

FIG. 41 shows the shear-thinning of dSIS-NB bioink. The viscosity of all dSIS-NB samples at 25° C., spanning different concentrations ranging from 0.8 to 4.5 wt %, exhibited a rapid decline as the shear rate increased from 10⁻¹ to 10⁻².

FIG. 42 shows high printability of dSIS-NB bioink at two different concentrations resulting in the formation of 3D square grids.

FIG. 43 shows a tube-shaped printed object with 4.5 wt % dSIS-NB bioink.

FIG. 44 shows in situ photorheometry of dSIS-NB gelation with tartrazine added as a photoabsorber to improve printing fidelity. 0.8 wt % dSIS-NB, 0.8 wt % PEG4SH, and 5 mM LAP. 405 nm at 28.8 mW/cm².

FIG. 45 shows a computer-aided design of a star-shaped object for DLP bioprinting.

FIG. 46 shows the DLP printed dSIS-NB gel. 2 mM tartrazine was added.

FIG. 47 shows a representative live/dead confocal image of interconnected microvascular HUVEC network within the printed hydrogel on day 3.

### DETAILED DESCRIPTION

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

The singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. In some embodiments, the term “about” in reference to a number or range of numbers is understood to mean the stated number and numbers+/−10% thereof, or 10% below the lower listed limit and 10% above the higher listed limit for the values listed for a range.

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

In general, this disclosure involves a composition with dECM functionalized with one or more norbornene substituents. As described further herein, these norbornene-functionalized dECM compositions may be crosslinked to provide a biologically compatible hydrogel. In particular, in some embodiments, the norbornene-functionalized dECM may be photocrosslinked with a thiol-bearing compound.

In one aspect, this disclosure provides a synthesis method for a photocrosslinkable macromer that is significantly more efficient than, and that avoids the use of noxious chemicals that are used in, previously known processes. In one embodiment, a method for synthesizing the macromer includes reacting decellularized extracellular matrices (dECM) with carbic anhydride and a nucleophilic catalyst to yield a norbornene-functionalized dECM. The dECM includes a plurality of amine functional groups. Carbic anhydride is an odorless, norbornene-derivative containing anhydride, which has shown promise as an agent for preparing norbornene functionalized materials.

In some embodiments, the dECM comprises decellularized liver, decellularized lung, decellularized pancreas, decellularized kidney, decellularized muscle, decellularized cardiac matrix, dSIS, or the like.

In some embodiments, the nucleophilic catalyst comprises a pyridine-based catalyst or a tertiary amine-based catalyst. In some embodiments, the nucleophilic catalyst comprises a member selected from the group consisting of pyridine, p-pyrrolidinopyridine, 4-dimethylaminopyridine, trimethylamine, triethylamine, diisopropylethylamine. In some embodiments, the nucleophilic catalyst comprises 4-dimethylaminopyridine.

Another unique feature of the methods described herein lies in the fact that the norbornene functionalized macromer produced by the reaction includes a norbornene domain that includes a carboxyl group. One advantage of having a carboxyl group on the norbornene domain of a macromer synthesized in accordance with this disclosure is that the resulting macromer, referred to herein as a dECM-NB macromer, has unexpected and advantageous properties. For example, when used to produce a hydrogel as described herein, a dECM-NB macromer produces a hydrogel that is significantly more labile to hydrolytic degradation, i.e., that degrades much more quickly upon exposure to water than other macromers previously described. As will be appreciated by a person of ordinary skill in the art, a higher the concentration of dECM-NB macromers used in the reaction mixture produces a hydrogel having higher hydrolytic degradation kinetics than does a hydrogel produced from a reaction mixture having a lower concentration of dECM-NB macromers.

In another aspect of the disclosure, there is provided a method for making a hydrogel that includes: reacting decellularized extracellular matrices (dECM) with carbic anhydride and a nucleophilic catalyst to yield a norbornene-functionalized dECM; and reacting the norbornene-functionalized dECM with at least one thiol-bearing compound. The proportions of the norbornene-functionalized dECM and the thiol-bearing compound to one another may be determined based upon the stoichiometric proportions of reactive groups necessary to optimize the reactivity of the reactants, and other practical considerations that are well within the purview of a person of ordinary skill in the art.

In some embodiments, the thiol-bearing compound comprises a multi-functional thiol. In some embodiments, the thiol-bearing compound comprises a member selected from the group consisting of 1,4-dithiothreitol, 4-arm thiolated PEG, a peptide that includes more than one cysteine residue, a natural or thiolated protein such as thiolated gelatin, thiolated hyaluronic acid, and thiolated collagens, and combinations thereof. In some embodiments, the thiol-bearing compound comprises dithiothreitol.

In addition to the methods described above, in another aspect, the disclosure provides hydrogel materials produced as described herein. In some embodiments, the hydrogel materials are provided as bulk materials. In some embodiments, the hydrogel materials are contained within packages or containers. In some embodiments, the hydrogel materials are contained within packages or containers as a part of a kit that includes one more additional elements. In some embodiments, the hydrogel materials are contained within packages or containers with information in printed form or recorded in an electronic medium. In some embodiments, the information includes instructions for using the hydrogel materials. In some embodiments, the information includes a description of properties or characteristics of the hydrogel material contained within the package or container.

In other aspects of the disclosure, there are provided a wide variety of methods for using a hydrogel material made in accordance with this disclosure including, for example and without limitation, in biomaterials and tissue engineering applications. The resulting dECM-NB hydrogels preserve microscopic fibrils of the dECM, while exhibiting high viscoelasticity and tunable stiffness. Compared with non-functionalized dECM or even Type I collagen hydrogels, the new dECM-NB hydrogels significantly promote in vitro vasculogenesis and angiogenesis of human umbilical vein endothelial cells, and substantially boost the migration of pancreatic cancer cells when co-cultured with human pancreatic cancer-associated fibroblasts (CAFs). Moreover, dECM-NB functions not only as a shear-thinning bioink compatible with extrusion-based 3D bioprinting but also serves as a photocrosslinkable bioresin for 3D DLP printing and microporous annealed particle (MAP) hydrogel forming. This enables the fabrication of intricate cell-laden volumetric structures, contributing to advanced tissue engineering applications.

Potential uses of such hydrogels include their use as templates for 3D culturing of cells, such as multipotent mesenchymal stem cells. Multipotent mesenchymal stem cells (MSCs) have high potential for many cell-based therapies as they can be isolated from multiple sources (e.g., bone marrow, umbilical cord, adipose tissue, etc.) and be readily differentiated into adipocytes, osteoblasts, and chondrocytes. MSCs can also undergo transdifferentiation into cardiomyocytes, neurons, and corneal epithelium cells. Additionally, studies have found that the secretory factors of MSCs can be utilized for regulating inflammatory and immune responses. Notably, MSC secretome can be manipulated by controlling matrix stiffness, viscoelasticity, and the presence of biological motifs (e.g., fibronectin). Many MSC-secreted factors, including transforming growth factor-β (TGF-β), insulin-like growth factor (IGF), and fibroblast growth factor-2 (FGF-2), have been shown to stimulate cell proliferation, migration, and differentiation. Furthermore, studies have found that, compared with 2D culture, MSCs cultured in 3D spheroids promoted cell proliferation, secretory properties, cell-cell interactions, and in vivo survival. In particular, a 2D surface un-naturally polarizes cells and lacks characteristics/architectures of a native cellular microenvironment needed to maintain the phenotype of the isolated cells. Studies have also revealed that assembling MSCs into multi-cellular spheroids increased the level of cell-cell interaction, leading to dramatically improved secretory properties and differentiation potential towards certain lineages (e.g., hepatocytes, osteoblasts, and neurons). MSCs have also been reported to exhibit anti-cancer effects via enhancing AKT signaling.

3D hydrogel scaffolds with physicochemically relevant properties have increasingly been used for MSC culture. Unless using hydrogel amenable to controllable degradation, chemically crosslinked hydrogels generally have nanometer-scale pores (or mesh) that will impede macromolecular diffusivity. Additionally, MSCs do not naturally form spheroids in 3D hydrogels, hence the encapsulated MSCs would remain disperse within the hydrogel matrix. The most efficient method to generate MSC clusters is by using well-plates with a hydrophobic surface and/or with special geometry (e.g., AGGREWELL™ plate). After assembly, MSC spheroids are collected and encapsulated in a hydrogel matrix to provide the cells with desired cell-matrix interactions. However, the handling of MSC spheroids may be challenging and the distribution of MSC spheroids within the bulk hydrogels may be non-uniform due to the settlement of the multicell spheroids. Furthermore, very few methods permit the generation of hollow cell spheres with a lumen structure, such as that found in ocular lens and lung alveoli. Therefore, a robust and powerful material engineering platform is needed to assist structured MSCs assembly in situ.

Hydrogels with macroscopic pores (i.e., macroporous hydrogels) are ideal for many biomedical applications owing to their improved transport properties and opportunity for enhanced cell-cell interactions within the matrix. Given the biphasic nature of the macroporous hydrogels, extracellular properties of the encapsulated cells could be decoupled from the bulk hydrogel matrix characteristics. As such, cellular assembly, attachment, and differentiation could be controlled by tuning extracellular properties independent of hydrogel stiffness, viscoelasticity, and cell adhesiveness.

A number of approaches have been employed to fabricate macroporous hydrogels, including salt leaching and gas forming, which depend on embedding and removing porogens from the crosslinked hydrogels. While this approach is straightforward, commonly used porogens (e.g., NaCl crystals and CO₂) are not compatible with in situ cell encapsulation. Furthermore, post-synthesis cell seeding is required but the passive penetration of cells into the porous hydrogel may be challenging, resulting in non-uniform cell distribution. Alternatively, cell-laden microgel templating within a continuous matrix could directly introduce cells into a 3D environment and cell structures could be formed in situ. But sacrificial materials (e.g., pH, photo, enzyme or thermal sensitive) are either limited in cytocompatibility or their degradation products (e.g., gelatin-derived peptides) may alter stem cell signaling pathways. Even though gelatin and alginate based hydrogels/microgels offer good cytocompatibility to the encapsulated cells, their dissolution requires changing temperature (for gelatin) or using external molecular trigger (e.g., metal ion chelators).

Recently, microporous annealed particle hydrogels (MAP) have become an attractive strategy where MSCs were seeded and attached on the annealed microgel interfaces. However, current MAP scaffolds cannot support MSC assembly into spheroids. Alternatively, hydrogels susceptible to hydrolytic degradation could be harnessed for forming dissolvable microgels to template the otherwise nanoporous hydrogels into macroporous gels. For example, microgels crosslinked from poly(ethylene glycol) (PEG) diacrylate (PEGDA) and dithiol crosslinker have been templated as sacrificial phase. The ester bonds in crosslinked microgels are labile toward hydrolysis for fabricating macroporous hydrogels. However, conventional ester hydrolysis induced gel degradation is a slow process that precludes the applications of such a platform in sensitive cell candidates that rely on cell-cell interaction to survive and function.

In some embodiments, droplet-microfluidics may be employed to fabricate cell-laden dissolvable microgels with well-controlled size and degradation profiles. These cell-laden dissolvable microgels may then be loaded into another bulk hydrogel. Macroporous hydrogels may then be formed by allowing the microgels to dissolve, leaving behind the cells in the pores. By tuning cell adhesiveness of the continuous matrix, multi-cellular structures may be formed.

In embodiments, the multi-cellular structures may include pancreatic cancer cells COLO-357, lung epithelial cells A549, mouse MSCs, human MSCs, or a combination of two or more of these. Besides morphological tunability, different assembly structures may also affect MSC therapeutic potential. This effect may be investigated by analyzing phosphorylation of a panel of AKT signaling proteins or the secretion of growth factors and cytokines.

The processes and hydrogel materials in accordance with this disclosure also provide for the ability to tune hydrogel degradation kinetics. In one aspect of the disclosure, hydrogel materials are synthesized in a controlled manner to have pre-engineered hydrolytic degradation profiles tuned for a particular use within a range of desired properties. The profiles are tuned by selection of functional moieties within the macromers and/or by selection of various combinations of macromers and proportions of macromers within such combinations and/or by selection of dithiols having different properties for cross-linking the macromers to form the hydrogel materials. In some embodiments, a method for tuning hydrogel degradation kinetics includes: (i) mixing at least two norbornene-functionalized macromers, at least one of the norbornene-functionalized macromers dECM-NB macromer; (ii) controlling the proportions of the macromers and/or dithiol based on at least one desired hydrogel characteristic selected from the group consisting of cross-link density, hydrolytic degradation kinetics, shear modulus and swelling ratio; (iii) mixing the macromers with a predetermined quantity of dithiol molecules to provide a reaction mixture; and (iv) applying ultraviolet radiation to the reaction mixture to initiate a photo-gelation reaction to yield a hydrogel having tuned degradation kinetics. In some embodiments, the method also includes selecting the dithiol molecules based on a functional characteristic of the dithiol molecules that modifies the degradation kinetics of the hydrogel.

Aspects of the present disclosure can be described as embodiments in any of the following enumerated clauses. It will be understood that any of the described embodiments can be used in connection with any other described embodiments to the extent that the embodiments do not contradict one another.

In an aspect, either alone or in combination with any other aspect, a composition comprises decellularized extracellular matrices, wherein the decellularized extracellular matrices comprise one or more norbornene substituents.

In an aspect, either alone or in combination with any other aspect, the one or more norbornene substituent is attached to one or more primary amines of the decellularized extracellular matrices.

In an aspect, either alone or in combination with any other aspect, the decellularized extracellular matrices are derived from a mammal.

In an aspect, either alone or in combination with any other aspect, a macromer for producing a hydrogel comprises decellularized extracellular matrices, wherein the decellularized extracellular matrices comprise one or more norbornene substituents.

In an aspect, either alone or in combination with any other aspect, a method for making a photocrosslinkable macromer comprises reacting decellularized extracellular matrices (dECM) with carbic anhydride and a nucleophilic catalyst to yield a norbornene-functionalized dECM.

In an aspect, either alone or in combination with any other aspect, the dECM comprises one or more primary amines.

In an aspect, either alone or in combination with any other aspect, the photocrosslinkable macromer comprises one or more amide linkages formed by the one or amines of the dECM and a carboxyl domain of the norbornene.

In an aspect, either alone or in combination with any other aspect, the nucleophilic catalyst comprises triethylamine.

In an aspect, either alone or in combination with any other aspect, a method for making a hydrogel comprises reacting decellularized extracellular matrices (dECM) with carbic anhydride and a nucleophilic catalyst to yield a norbornene-functionalized dECM; and reacting the norbornene-functionalized dECM with at least one thiol-bearing compound.

In an aspect, either alone or in combination with any other aspect, the reacting the norbornene-functionalized dECM with at least one thiol-bearing compound comprises exposing the norbornene-functionalized dECM and the thiol-bearing compound to electromagnetic radiation having a wavelength from 365 nm to 405 nm.

In an aspect, either alone or in combination with any other aspect, the thiol-bearing compound is selected from the group consisting of 1,4-dithiothreitol, 4-arm thiolated PEG, a peptide that includes more than one cysteine residue, a natural or thiolated protein, thiolated gelatin, thiolated hyaluronic acid, thiolated collagens, and combinations thereof.

In an aspect, either alone or in combination with any other aspect, the thiol-bearing compound comprises dithiothreitol.

In an aspect, either alone or in combination with any other aspect, the hydrogel comprises an orthogonal thiol-norbornene hydrogel.

In an aspect, either alone or in combination with any other aspect, a hydrogel is produced according to any method disclosed herein, including any combination of described steps to arrive at the hydrogel.

In an aspect, either alone or in combination with any other aspect, the hydrogel is used for a purpose selected from the group consisting of as a substrate for short-term 2D cell culturing, 3D in situ encapsulation of viable cells, controlled delivery of drugs and other bioactive materials, tissue engineering, tissue regeneration, creating micropores in a bulk polyethylene glycol-based hydrogel, creating a sacrificial material in a 3D bioprinted article, creating a sacrificial material in a photolithography-based biofabrication product, and creating a sacrificial bioink for extrusion-based or photolithography-based 3D bioproducts.

Further reference is made to the following experimental examples.

### EXAMPLES

The following examples are given for the purpose of illustrating various embodiments of the invention and are not meant to limit the present invention in any fashion. The present examples, along with the methods described herein are presently representative of preferred embodiments, are provided only as examples, and are not intended as limitations on the scope of the invention. Changes therein and other uses which are encompassed within the spirit of the invention as defined by the scope of the claims will occur to those skilled in the art.

Bovine dSIS was modified with norbornene moieties, creating a new photocrosslinkable macromer (i.e., dSIS-NB) with native characteristics favorable for advanced tissue engineering applications. Briefly, dSIS-NB was derived from reacting dSIS with carbic anhydride in a base catalyst triethylamine (TEA) buffer solution at room temperature. Hydrogel crosslinking was achieved by reacting dSIS-NB with PEG4SH via a step-growth thiol-norbornene reaction. The resulting dSIS-based thiolene hydrogels preserved microscopic fibrils of the SIS, with tunable stiffness. Compared with non-functionalized dSIS or even Type I collagen hydrogels, the new dSIS-NB hydrogels significantly promoted in vitro functional vasculogenesis and angiogenesis of human umbilical vein endothelial cells, and substantially boosted the migration of pancreatic cancer cells when co-cultured with CAFs. Moreover, dSIS-NB functions not only as a shear-thinning bioink compatible with extrusion-based 3D bioprinting but also serves as a photocrosslinkable bioresin for 3D DLP printing and MAP hydrogel forming. This enables the fabrication of intricate cell-laden volumetric structures, contributing to advanced tissue engineering applications.

### General Methods

Reagents and materials. Sodium dodecyl sulfate (SDS) (#L4390) was acquired from Sigma-Aldrich. Carbic anhydride (#129-64-6) and triethylamine (TEA) (#21951-0500) were obtained from Acros Organics. Ellman's reagent (#22582) was purchased from Life Technologies. FBS, P/S, tartrazine (#T0388) were acquired from Sigma-Aldrich.

4 arm-PEG (#P2003-131225, 20 kDa) and 4-arm PEGSH (#155-163,10 kDa) was obtained from JenKem Technology USA and Laysan Bio, respectively. Paraformaldehyde (PFA) (#158127), 4-(Dimethylamino)pyridine (DMAP) (#107700), lithium phenyl-2,4,6-trimethylbenzoylphosphinate (LAP, #900889-1G) and tartrazine (#T0388) were acquired from Sigma-Aldrich. RhB-PEG-SH (#PG2-RBTH-3k) was purchased from NANOCS. QK peptide (KLTWQELYQLKYKGIGC (SEQ ID NO.: 1), C-terminal Cysteine residue added for orthogonal thiol-norbornene conjugation) was custom synthesized and purified to at least 85% purity by GenScript Biotech. Bovine Serum Albumin (BSA) (#10842-770) was obtained from Avantor. Dulbecco's Phosphate Buffered Saline (DPBS, #21-031-CV) was acquired from Corning Life Sciences. All other reagents were obtained from Fisher Scientific unless otherwise noted.

dSIS-NB synthesis. The fresh bovine small intestine was purchased from a local food market and kept at −20° C. for use in 3 months. One thousand grams of intestine were washed carefully with tap water then cut to 10 cm in length and the mesenteric tissues were removed. The intestine segments were flipped inside out and scrubbed to remove mucosal epithelium and lamina propria afterward, flipping them back to the original shape, tunica serosa and tunica muscularis externa were then removed. The SIS was sterilized with 0.1% peracetic acid for 15 min and stored at −80° C. until use within 1 month. Before decellularization, the SIS tissues were washed in phosphate buffered saline (PBS) 5 times and then stirred in PBS containing 1% SDS, 2% Penicillin-Streptomycin, and gentamycin (0.5 mg/mL) for 3 days (change buffer every 12 hours). The tissues were then treated with 1% triton-X for 24 h prior to vigorous washing in deionized water for 72 h (change every 6-8 hours) then lyophilized for 72 h under −50° C., 20 Pa. The desiccated tissues were ground using a blender and kept at −80° C. for use within 12 months.

100 mg of dSIS powder were digested in an acidic pepsin solution containing 10 mg of pepsin, 50 mL of 0.01 N HCl, pH 2 at room temperature (RT) for 4 days (0.2 wt. %). The acidic digested dSIS was then dialyzed against pre-cooling sterile deionized (DI) water at −4° C. for 4 days to remove pepsin and HCl followed by lyophilization for 3 days. This dried sponge-form dSIS was dissolved in sterile PBS at 4° C. for control conditions. For modification with norbornene, 70 mg (mass ratio MR_{[CA/dSIS]}=0.7) or 100 mg (mass ratio MR_{[CA/dSIS]}=1) of carbic anhydride (CA) were added to the 50 mL of 0.2 wt % acidic dSIS solution supplemented with about 70 μL (for mass ratio MR_{[CA/dSIS]}=0.7) or 100 μL (for mass ratio MR_{[CA/dSIS]}=1) of TEA with magnetic stirring. Dropwise added TEA (10 μL) until the reacting solution became transparent. The reaction was conducted at 25° C. with 400-rpm stirring for 4 h then the solution was passed through a 40-μm cell strainer to remove debris. The reacted solution was dialyzed against pre-cooling autoclaved DI water at 4° C. for 3 days using a 10-12 kDa dialyzing bag with frequent changes of fresh autoclaved DI water every 24 h followed by lyophilization for 3 days. The dried dSIS-NB then was dissolved in PBS using a vigorous votexer at 4° C., stored at 4° C., and used within one week.

Analysis of dSIS-NB functionalization. Nuclear magnetic resonance (NMR) spectroscopy was used to determine the presence of norbornene in the structure of dSIS after modification. dSIS and dSIS-NB (high CA and low CA) were dissolved at 0.5 wt % in deuterated water with 10 mM deuterium hydrochloric acid and analyzed by ¹H NMR.

Ellman's assay was employed to indirectly assess the degree of norbornene functionalization achieved in dSIS-NB. This was accomplished by quantifying the concentration of thiol groups present in the samples. 100 μL of Ellman's reagent (5,5′-dithiobis-(2-nitrobenzoic acid), or DTNB) was added to 5 mL of Ellman's buffer (0.2 M Na₂HPO₄+0.2 M EDTA). Two hundred μL of 6 mM L-cysteine (L-cys) were prepared in PBS followed by seven 2-fold serial dilutions to form a range from 0.046875 mM to 6 mM of L-cys concentration. PBS was used as the blank. A solution containing 6 mM L-cysteine, 5 mM LAP, and 1 wt % dSIS or dSIS-NB was subjected to light exposure under a 365 nm light source for 1 min. Twenty-five μL of samples were placed in triplicate into a 96-well plate followed by the addition of 200 μL Ellman's reagent buffer. The absorbance of the samples was then measured using a 405 nm microplate reader (Synergy HT, Biotek). The degree of norbornene functionalization was understood as the reacted amount of L-cys in the solution once applying a blank level with PBS.

Rheolygical and microstructure analysis. A disk-shaped hydrogel was formed by applying 40 μL of precursor solution onto water-repellent-treated glass slides and then exposed under 365 nm light (2.9 mW/cm²) for 2 min. This process resulted in a hydrogel with an approximate diameter of 8 mm and a thickness of 0.8 mm, which was subsequently used for rheological characterization of the hydrogels. The moduli of the bulk hydrogels were measured using the modular compact rheometer (MCR 102, Antor Parr) with a strain sweep normal force (NF) control mode. The temperature, NF, shear strain, and frequency for the measurement were 25° C., 0.25 N, 0.1-0.5%, and 1 Hz, respectively. The shear modulus (G) was utilized as a quantitative measure of gel stiffness. The shear modulus was calculated using G=√(G′²+G″²). The values of storage modulus (G′) approximate that of G, as the loss modulus (G″) was significantly lower than that of G′ due to the elastic nature of the hydrogels. For degradation test, bulk hydrogels were incubated in sterile cell culture media containing high glucose DMEM, 10% FBS, 1% P/S at 37° C., 5% CO₂. The viscoelasticity curves were acquired using stress relaxation mode with an initial strain of 5% for a total 2000 second acquisition time.

Scanning electron microscope (SEM) was used to show the microstructure of dSIS and dSIS-NB hydrogels. Bulk hydrogels were washed in deionized water for 30 min to remove salt and residual chemicals before freezing at −80° C. and lyophilizing for 24 h under 20 Pa, −50° C. Freeze-dried samples were torn down and metal coated with an Emitech K575X sputter coater (Quorum Technologies Ltd., United Kingdom). Microstructural images were acquired using a JEOL JSM-7800F.

Cell culture and encapsulation. HUVECs (cryopreserved) and endothelial cell media kits were purchased from ScienCell Research Laboratories. Pancreatic cancer cells COLO-357 were obtained from Dr. Murray Korc's lab and were stably transduced with mKate2, a red fluorescent protein (RFP), using Incucyte® Cytolight Lentivirus reagent, followed by puromycin selection. Immortalized human pancreatic cancer-associated fibroblasts (1303-GFP-49-hT or CAF), which were labeled with green fluorescent protein (GFP), were obtained from Dr. Melissa Fishel's laboratory at Indiana University School of Medicine. HUVECs were thawed and cultured for 2-3 days in vascular cell basal media supplemented with endothelial cell growth kit-VEGF and antibiotics at a density of 5,000-7,500 cells/cm² until they reached about 60% to 70% confluency. COLO-357s and CAFs were thawed and expanded in high-glucose DMEM containing 10% FBS, 1% P/S. Confluent cells were detached using 0.05% trypsin-EDTA (#15400054, Life Technologies) for 2 min after washing twice with sterile PBS, harvested, and centrifuged at 500×g for 5 minutes. Cells at passage 3 to 9 were used for all experiments in this study.

For encapsulating single cells, HUVECs were suspended within precursor solutions at a density of around 5×10⁶ cells/mL. To create HUVEC spheroids, approximately 1500 cells were initially cultured in u-bottom 96-well microplates for two days. Following this, the spheroids were harvested, suspended in precursor solutions, and utilized for further procedures. To prevent the cell spheroids from sinking during gel formation, a glass-bottom petri dish (ø10 mm) was first coated with a thin layer of the same gel (30 μL). Subsequently, 60 μL of the cell precursor solution was applied to the top of this layer before proceeding with the crosslinking process.

To form a 3D microvascular network inside a u-slide for microparticle perfusion, we used a u-slide chemotaxis (80326, ibidi). HUVECs were suspended in a solution containing 0.8 wt % dSIS-NB, 0.8 wt % PEG4SH, and 5 mM LAP at 107 cells/mL of cell density and then injected into the middle chamber of the u-slide and exposed to 365 nm light (2.9 mW/cm²) for 2 min. HUVEC culture media was contained in two side chambers of the u-slide and refreshed every 8 h. On day 3, one side chamber was used to load new cell media containing fluorescent microparticles (ø2 or 10 μm, F8853, 09-980-514, Fisher Scientific). Another side chamber was empty to generate interstitial flows within the microvascular network. Time-lapse imaging was performed using a 3D digital benchtop confocal microscope (BC43, Andor).

To generate micro spheroids of COLO-357 and CAF cells, about 400 CAF cells were employed to form each CAF or CAF-COLO-357 spheroid. Similarly, about 400 COLO-357 cells were utilized for creating each COLO-357 spheroid, while about 80 COLO-357 cells were employed for the formation of each CAF-COLO-357 spheroid. Spheroids were cultivated for a duration of 2 days within a concave agarose 3D-culture microarray (16×16 array), which was constructed using a specialized 3D petri dish provided by MicroTissues Inc. Micro spheroids were collected and suspended in precursor solution at around 100 spheroids/mL of density. The cell culture media were changed by 60% every 24 h.

Bioprinting. In the context of extrusion-based bioprinting, bioinks comprising 3 wt % and 4.5 wt % dSIS-NB, along with 1 wt % PEG4SH and 5 mM LAP, were formulated within a 3 mL syringe. The printability of the dSIS-NB bioink was assessed through extrusion-based methods under ambient conditions at 25° C. using a 3D printer (Cellink, BioX). A 22-gauge blunt needle was used as the printing nozzle, operating under an extrusion pressure of 20-30 kPa and a printing speed of 1.5 mm/s. Crosslinking was carried out after every 2 layers using the built-in 365 nm light source. The exposure time was set at 5 seconds, and a distance of 5 cm was maintained between the sample and the light source during the crosslinking process. The designed printed objects were created and then exported to STL files using the TinkerCad website.

Additionally, a digital light processing (DLP) bioprinter (LumenX+, Cellink) was employed to fabricate dSIS-NB hydrogels, demonstrating the capability of dSIS-NB bioink for DLP-based printing. Printing z-step, wavelength, light intensity, and exposure time were set up at 100 μm, 405 nm, 28.8 mW/cm², 13 sec, respectively. Tartrazine was employed as a photosensitive absorber to enhance the printing resolution. The impact of tartrazine on gelation kinetics of dSIS-NB was examined via in situ photopolymerization using the modular compact rheometer. Specifically, precursor solutions were prepared by mixing pre-determined concentrations of 0 mM, 2 mM, and 2.5 mM tartrazine, 10 mM mM LAP, 0.8 wt % dSIS-NB, and 0.8 wt % PEGSH in PBS. Fifty microliters of the solution were loaded between the measuring cylinder (8 mm diameter) and the platform of the rheometer. The gap between the plate and the cylinder was set at 100 μm. This value was equivalent to the step size in DLP printing. The strain and frequency of the measurement were set at 1% and 1 Hz, respectively. The printing z-step, wavelength, light intensity, and exposure time were configured for the condition involving 2 mM tartrazine: 100 μm z-step, 405 nm wavelength, 28.8 mW/cm² light intensity, and 8 seconds exposure time.

Microporous annealing particle gel. Microgels were generated using the emulsification technique under a sterile condition. Briefly, a precursor solution containing 2 wt % dSIS-NB, 0.65 wt % PEG4SH, and 5 mM LAP was vigorously vortexed with 2 wt % Pico-surf in Novec 7500 (at a 3:1 ratio of precursor and oil) and immediately exposed under 365 nm light, with a light intensity of 2.9 mW/cm² for 2 min. Microgels were collected by using a 70-μm cell strainer under 3000 rpm centrifugation in 1 min. Then the microgels were washed and centrifuged 5 times with PBS to remove residual oil.

MAP gels was created by incubating microgels with 0.5 wt % PEG4Tz (synthesized using a published protocol) for 30 min at 37° C. To visualize the microporosity of the MAP gels, the MAP gel was incubated with high molecular weight fluorescein isothiocyanate-labeled dextran (FITC-dextran, 2 MDa @ 1 mg/mL) then the MAP gels were observed under FV100 confocal microscope (Olympus, Japan).

Staining and imaging. For live/dead staining (Viability/Cytotoxicity assay kit for animal live & dead cells) images, samples were stained in 1×DPBS solution containing 2 μM calcein AM (stains live cells) and 4 μM EthD-III (stains dead cells) for 45 min at 37° C. The labeled cells were imaged by a confocal microscope (FV1000, Olympus, Japan). Calcein can be imaged using an Alexa 488 filter set, and EthD-III can be imaged using TexasRed filter sets.

Statistical analysis. The results are presented as the means±SEM. Comparisons between the two groups were performed using a two-tailed Student's t-test and One-way ANOVA in Prism 6.01 (GraphPad, USA), and a value of p<0.05 was considered statistically significant. Finally, the sample sizes are noted in the figure captions.

### Example 1

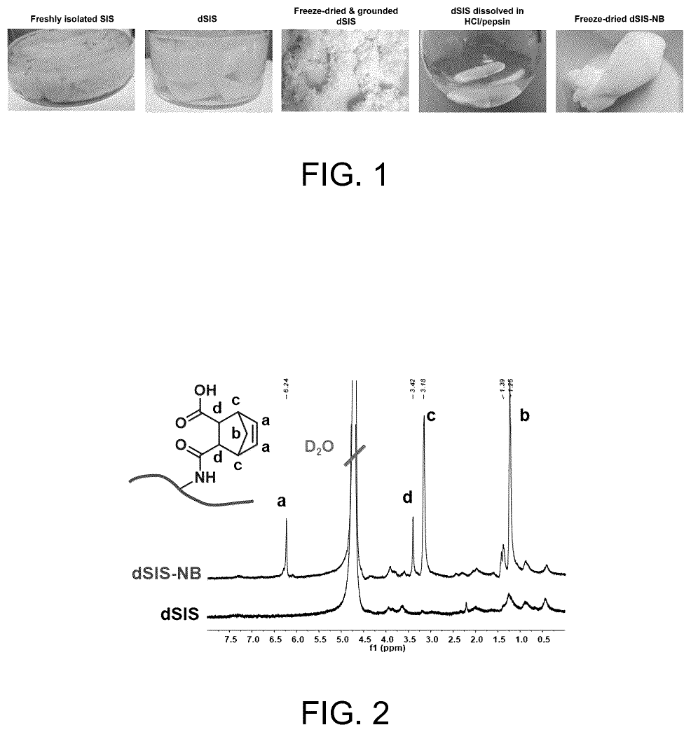

Synthesis of dSIS-NB. Small intestine submucosa was decellularized via washing/incubating the freshly isolated tissue in sodium dodecyl sulfate (SDS) solution containing Penicillin-Streptomycin and gentamycin for three days. The decellularization process led to gradual tissue whitening and reduction of surface wrinkles (FIG. 1). SDS is a potent agent for decellularization of heart, lung, kidney, liver, SIS, and cornea. To reduce the potential cytocompatibility issue, the decellularized SIS was thoroughly washed with 1% triton-X for 24hours, followed by vigorous washing in sterile deionized water for 72 hours (water changes every 6-8 hours). Next, the dSIS was lyophilized and blended into white powders (FIG. 1), which could be gradually dissolved in an acidic pepsin-containing solution (FIG. 1). dSIS contains abundant primary amines that serve as nucleophiles for reaction with carbic anhydride to form amide-linked norbornene (i.e., dSIS-NB). Prior to norbornene functionalization, the primary amine content on dSIS was quantified by fluoraldehyde assay and determined to be 2.94 mM per wt % of dSIS (Table 1). Triethylamine (TEA) was used as the base catalyst, as it was demonstrated to be a more effective catalyst than NaOH in the synthesis of GelNB. Carbic anhydride was added at an equal mass as dSIS. After reaction, dSIS-NB was dialyzed and freeze-dried to yield a loss sponge (FIG. 1). The norbornene functionalization was confirmed by ¹H NMR spectroscopy (FIG. 2). Specifically, the resonances in the range of approximately 6.0 to 6.2 ppm were assigned to the alkene protons within the carbic group. norbornene concentration was also quantified indirectly through a modified Ellman's assay where thiol-bearing molecule (i.e., cysteine) reacts specifically and rapidly with norbornene groups via a non-gelling thiol-norbornene photo-click reaction. The concentration of NB was determined to be 2.58 mM/wt % dSIS-NB.

TABLE 1

| Sample | Amine concentration (mM/wt %) |
|---|---|
| 1 | 2.88 |
| 2 | 3.02 |
| 3 | 2.72 |
| 4 | 3.14 |
| Average | 2.94 |

### Example 2

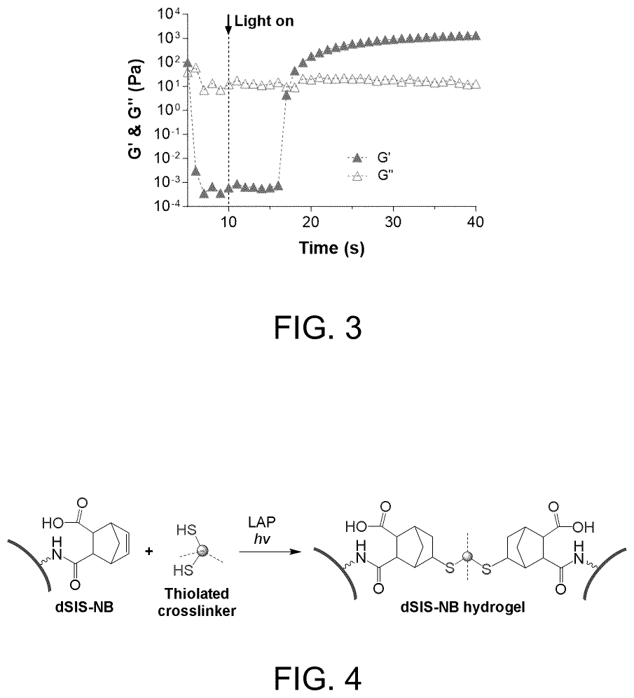

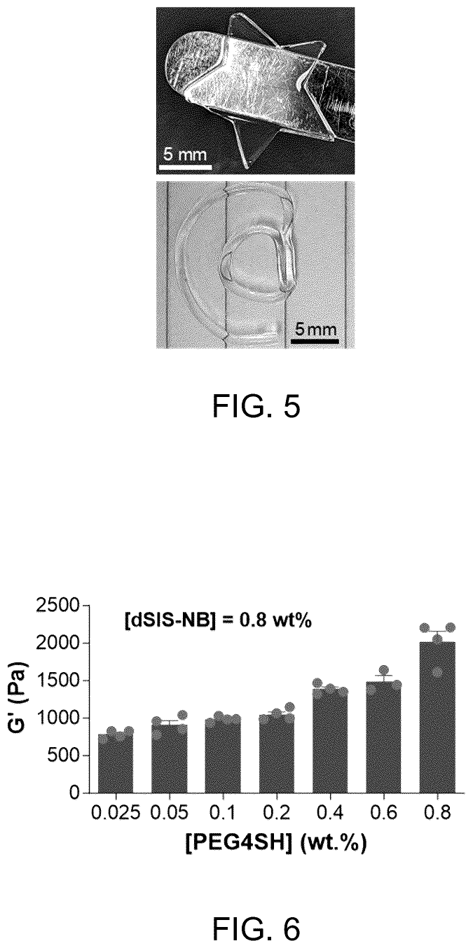

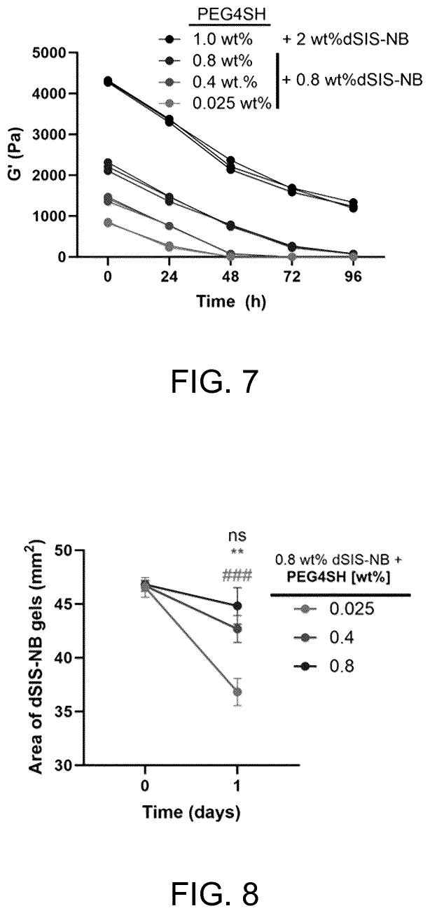

Characterization of dSIS-NB hydrogels. Like other norbornene-modified monomers, dSIS-NB could be cross-linked orthogonally with a wide range of thiol-bearing cross-linkers, including dithiothreitol (DTT), bis-cysteine bearing peptides, and thiolated macromers (e.g., PEG4SH). In the presence of a photoinitiator (e.g., lithium phenyl-2,4,6-trimethylbenzoylphosphinate or LAP), this gelation could be initiated under 365 nm or 405 nm light exposure (FIG. 4). For example, with a 365 nm light intensity of 2.9 mW/cm², rapid gelation of 0.8 wt % dSIS-NB (with 0.8 wt % PEG4SH and 5 mM LAP) using in situ photo-rheometry (FIG. 3) was observed. Specifically, the light-induced sol-gel transition occurred within 7 to 8 s and the shear moduli (G′) rapidly reached a stable plateau of around 2 kPa within 40 s. Light-mediated gelation permitted the formation of dSIS-NB hydrogels with various shapes and sizes (FIG. 5), including a hydrogel fiber. The rapid thiol-norbornene photo-gelation of dSIS-NB hydrogels was similar to that reported in the literature, offering numerous benefits for hydrogel scaffold fabrication and opportunities for various applications in tissue engineering, including vascularization and tissue regeneration. To formulate dSIS-NB hydrogels with tunable stiffness, 0.8 wt % dSIS-NB was mixed with varying concentrations of PEG4SH (0.025 wt % to 0.8 wt %) and the gelation was initiated by the same light exposure conditions (i.e., 5 mM LAP and 2 min of 365 nm light at 2.9 mW/cm²). Gelation occurred efficiently even at a low concentration of 0.025 wt % PEG4SH (FIG. 6), which corresponds to a thiol-to-norbornene ratio (R_{[SH/NB]}) of 0.048. The shear modulus measured approximately 800 Pa, making it a suitable stiffness for mimicking biological tissues, such as the spinal cord or brain. The stiffness of dSIS-NB hydrogels exhibited a notable increase at higher PEG4SH concentrations, ultimately reaching ˜2,200 Pa at a PEG4SH concentration of 0.8 wt % (resulting in an R_{[SH/NB]} of approximately 1.53). Hydrogel moduli could be further increased by using higher content of dSIS-NB (e.g., 2 wt % dSIS-NB with 1 wt % PEG4SH, leading to G′˜4,500 Pa. FIG. 7). The versatility of stiffness within the range of dSIS-NB hydrogels might make them suitable for a wide range of cell tissue engineering applications, both in vitro and in vivo. In addition, the dSIS-MA hydrogels created by Elomaa et al. swelled about 32 to 40% compared to their original size. This swelling could cause encapsulated cells to get far away from each other resulting in the loosening of the connection between cells to form functional tissues, especially vascularization. In contrast, the thiol-norbornene dSIS-NB hydrogels shrank their size by about 20.8, 8.6, and 4.2% for 0.8 wt % dSIS-NB crosslinked by 0.025, 0.4, and 0.8 wt % PEG4SH after 24 h, respectively (FIG. 8). Regardless their stiffness decreased (FIG. 7).

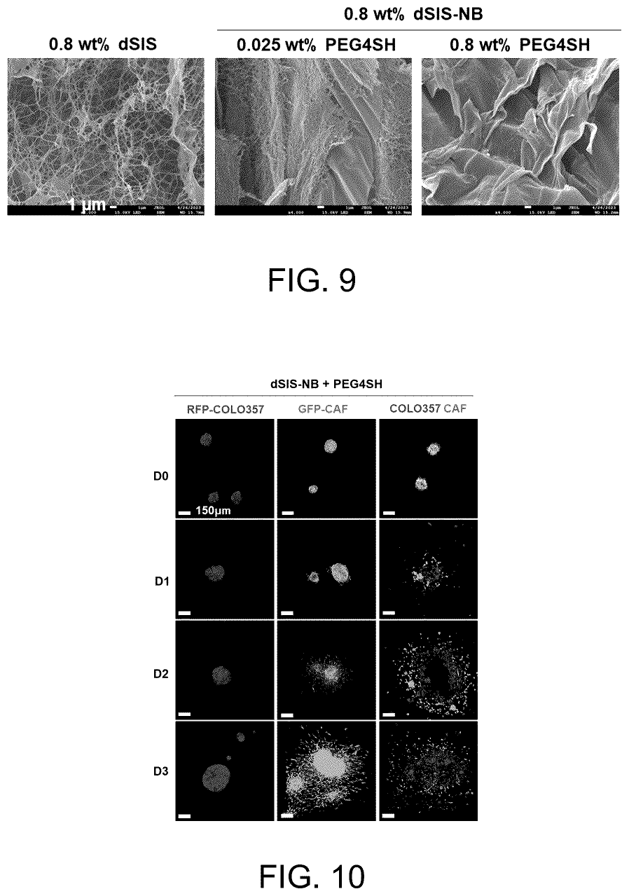

Next, the dSIS-NB hydrogel samples were examined with scanning electron microscope (SEM), with non-chemically crosslinked freeze-dried dSIS gel (37° C. for 30 min) as a control. SEM images revealed a network of interwoven fibers with diameters ranging from 80 to 160 nm (FIG. 9). Meanwhile, the freeze-dried dSIS-NB hydrogels crosslinked by 0.025 wt % PEG4SH also displayed some woven fibrillar structures (with fiber diameters of 44 to 83 nm) and areas of smooth aggregates. Upon increasing the PEG4SH concentration to 1 wt %, little fibers were observed and large areas with rough textures were notable. It is worth noting that these fiber dimensions do not represent the actual fiber size in swollen hydrogels as the samples were freeze-dried for SEM imaging. However, the presence of fibrils in the dSIS-NB hydrogels suggests that the modified macromers preserved a certain degree of collagenous structure. The fibrous structure may play a significant role in influencing cellular behaviors such as attachment, migration, interactions, proliferation, and differentiation.

### Example 3

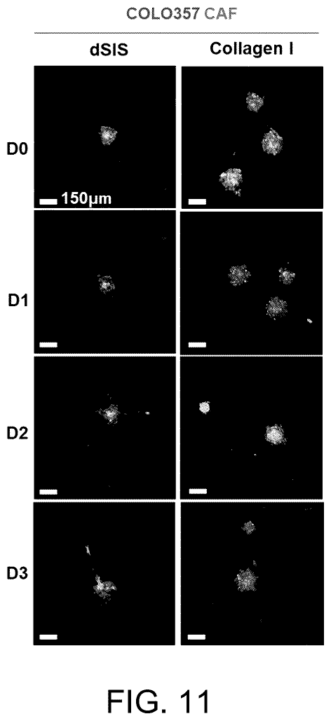

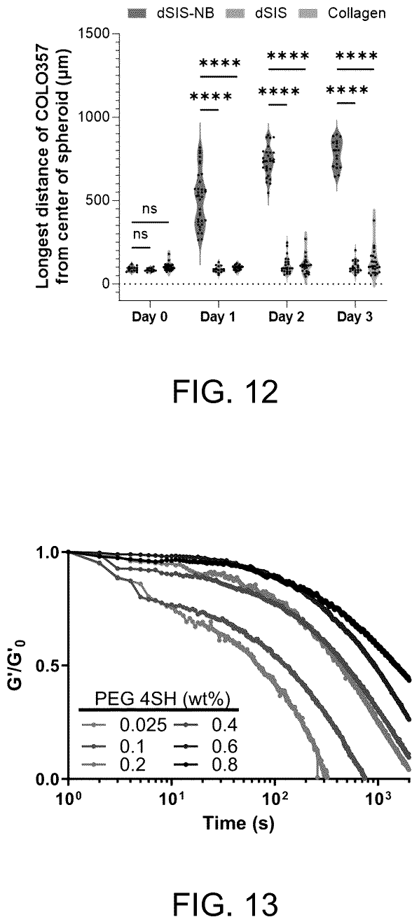

dSIS-NB hydrogels for in vitro cancer model. Encouraged by the rapid sprouting of vascular endothelial cells in orthogonally crosslinked dSIS-NB hydrogels, the ability to use this new hydrogel platform for studying cancer cell dissemination was determined. To this end, pancreatic cancer cell spheroids were generated from red fluorescent protein (RFP)-tagged COLO-357 (a pancreatic cancer cell line) and green fluorescent protein (GFP)-tagged patient-derived human pancreatic cancer-associated fibroblasts (CAF). RFP-COLO-357 and GFP-CAF spheroids were encapsulated in thiol-norbornene crosslinked dSIS-NB hydrogels, with physically crosslinked dSIS and Type I collagen gels as controls. Drastic differences in cell morphology were noted in the encapsulated cell spheroids. Specifically, RFP-COLO-357 spheroids gradually increased in size but showed limited cell spreading or invasion into the matrix (FIG. 10). On the other hand, GFP-CAF spheroids showed notable increases in both the spheroid size and individual cell sprouting from the spheroids (FIG. 10). Interestingly, mixed cell spheroids (i.e., single spheroids containing both RFP-COLO-357 and GFP-CAF) displayed fast cell spreading and invasion in dSIS-NB gels as early as 24 hours after encapsulation (FIG. 10). Conversely, mixed cell spheroids in physically crosslinked dSIS and Type I collagen gels exhibited limited morphological changes in 3 days (FIG. 11). The longest distance of RFP-COLO-357 cells from the center of the spheroids was further quantified. FIG. 12 shows that RFP-COLO-357 cells in dSIS-NB gels exhibited the most extensive spreading, covering an average distance of approximately 780 μm over a 3-day period. This was in stark contrast to that in dSIS and collagen gels, where the average longest distances were less than 200 μm. These differences could be attributed to the unique properties of dSIS-NB gels, such as their stiffness, nano-fibrous structure, and viscoelastic characteristics, as illustrated in FIGS. 3, 6, 7, 9, and 13. These properties likely played a collective role in promoting cancer cells spreading (FIG. 10).

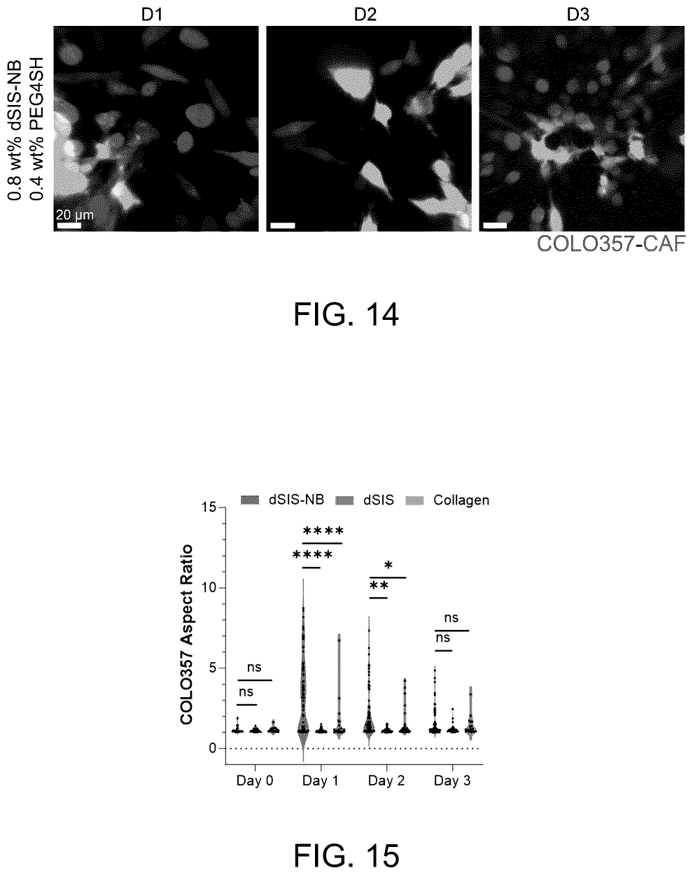

In mono-culture COLO-357 spheroids, cellular interactions were absent due to the lack of CAFs. Consequently, COLO-357s retained their spherical morphology but grew to larger sizes (FIG. 10). In contrast, when co-cultured with CAFs and encapsulated within dSIS-NB gels, cellular signaling between two kinds of cells could occur. CAFs migrated from the spheroids into the bulk gel concurrently attracted COLO-357s to migrate. Signaling molecules such as transforming growth factor-β (TGF-β) and fibroblast growth factor (FGF) need to be detected and quantified to have a deeper insight into this interaction which might contribute to the epithelial-mesenchymal transition (EMT) of COLO-357s. This transformation granted the cells mobility and the ability to migrate. This interaction was might also appeared with COLO-357s and CAFs in the co-culture spheroids within the native dSIS and collagen gels, leading to the migration of a few number of migrating COLO-357s, as shown in FIG. 10. However, it was worth noting that the migration may have been influenced by the lower stiffness and swelling characteristics of the collagen and native dSIS gels. During EMT, cancer cells were known to change their morphology from circular to spindle-shaped or spindled. Here, spindle-shaped COLO-357s became more abundant after 24 hours of encapsulation, with a significant decrease in their numbers on days 2 and 3 (FIG. 14 and FIG. 15). This result suggested that the migration of COLO-357s may be constrained when cells are more distant from each other.

### Example 4

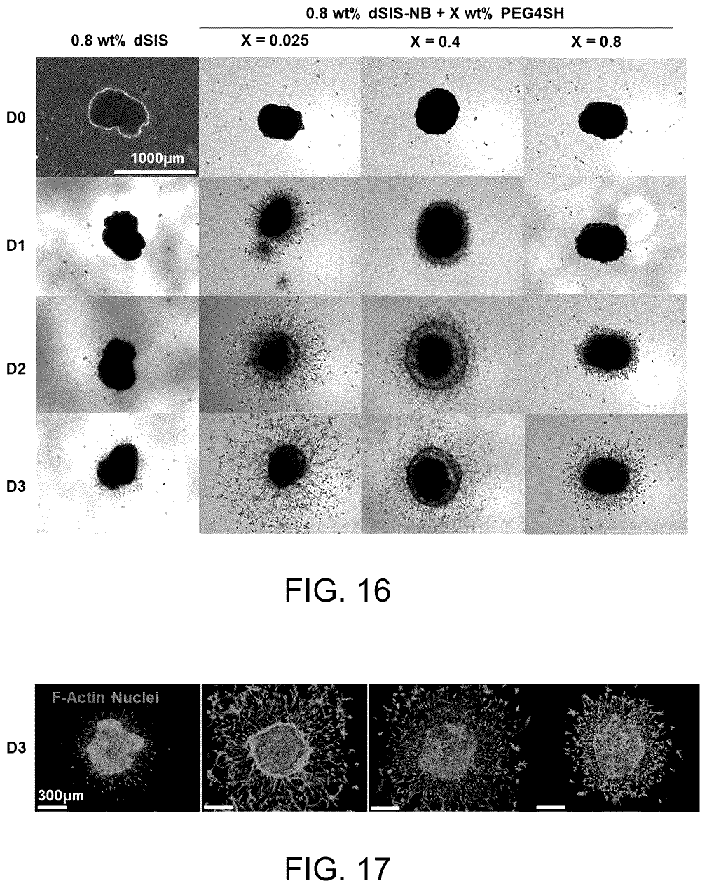

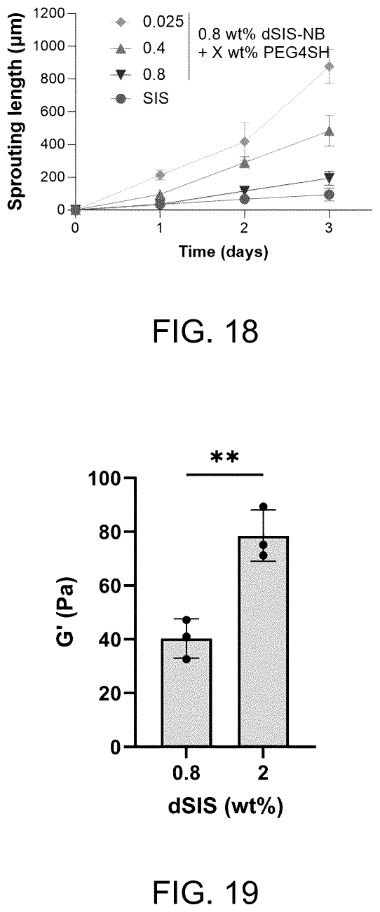

dSIS-NB hydrogels for functional vascularization and angiogenesis. To demonstrate the angiogenic properties of dSIS-NB hydrogels, HUVEC spheroids were generated and encapsulated in dSIS and dSIS-NB hydrogels. For HUVEC spheroids encapsulated in physically crosslinked dSIS hydrogels, cellular protrusion/sprouting was not visible in the first 24 h post-encapsulation. In contrast, significant protrusion along the periphery of the HUVEC spheroids was notable in all three dSIS-NB hydrogels, with the softest dSIS-NB gels (crosslinked by 0.025 wt % PEG4SH) HUVECs beginning to protrude after 3 to 6 h of encapsulation, producing the most sprouting (FIG. 16). The quantity and length of these sprouts were affected by the stiffness of the dSIS-NB hydrogels, with stiffer gels (i.e., higher concentrations of PEG4SH) supporting less sprouting. Results from F-actin staining 3D confocal images (FIG. 17) and quantitative analyses of sprouts (FIG. 18) demonstrated the same trend as the brightfield images. This vascular sprouting experiment demonstrated that the angiogenic properties of the new dSIS-NB hydrogels outperformed that of physically crosslinked dSIS matrices and likely also better than collagen-based hydrogels, such as those reported by Martina et al., Tetzlaff et al., and Mehes et al. Indeed, Martina et al. showed the lengths of sprouting of HUVECs in 100 to 200 μm using Type I collagen gel (0.12 wt %) after 48 h. On the other hand, Tetzlaff et al. and Mehes et al. showed that their HUVECs protruded 87 μm in collagen after 24 h and 400 μm in fibrin after 70 h, respectively. In contrast, the sprouting length of the HUVECs in the thiol-norbornene dSIS-NB hydrogels cross-linked by 0.025 wt % PEG4SH reached approximately 200 μm in 24 h and 950 μm after 72 h (FIGS. 16-18).

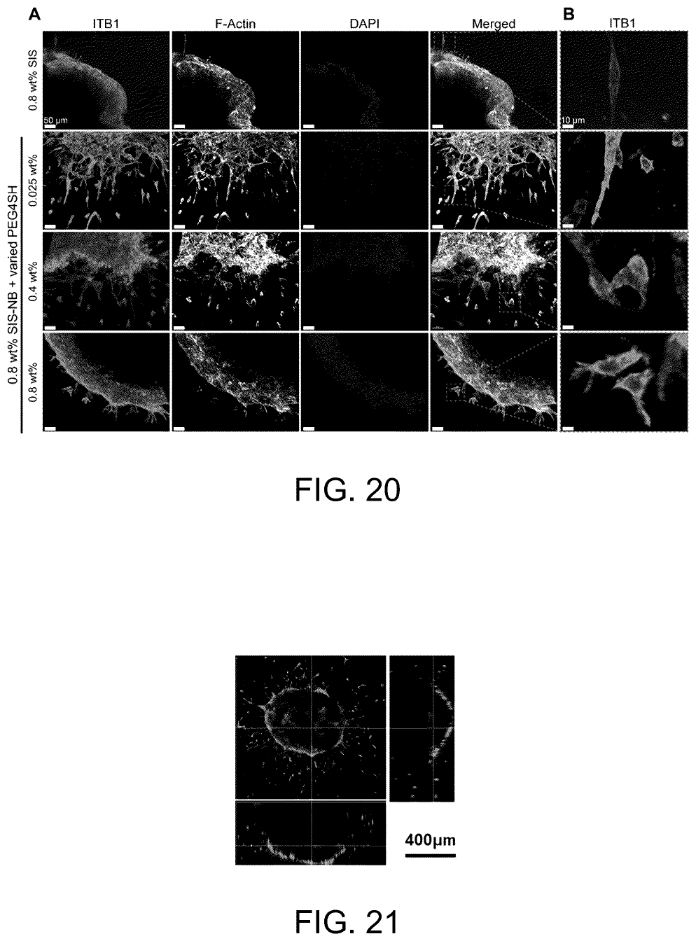

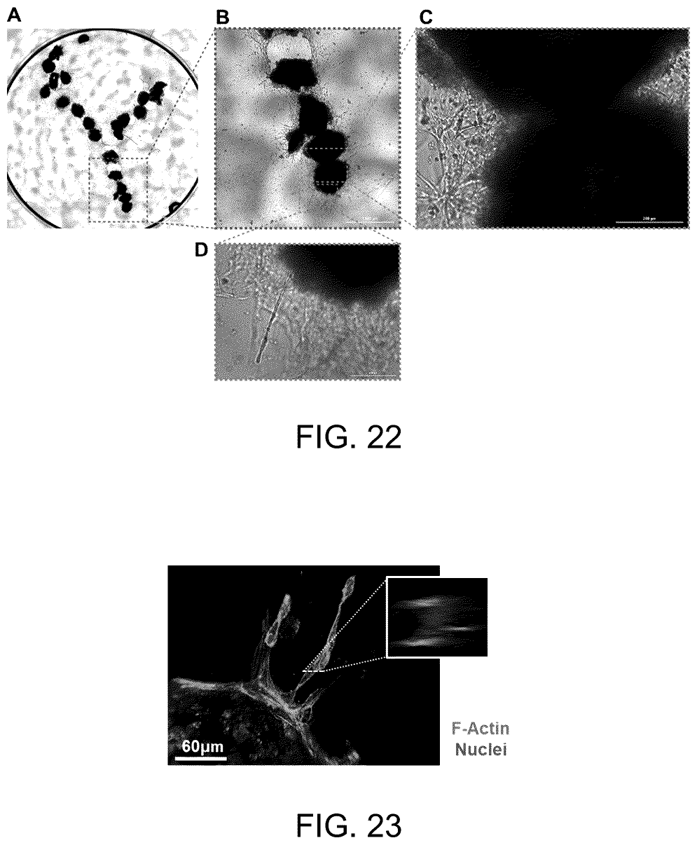

One notable observation from the sprouting experiments shown in FIGS. 16-18, 21, and 23 was that all three groups of thiol-norbornene dSIS-NB hydrogels outperformed the physically crosslinked dSIS hydrogels. Within the three dSIS-NB hydrogels, there was a negative correlation between the gel crosslinking density and the number/length of vascular sprouting. While physically crosslinked dSIS hydrogels were softer than even the softest dSIS-NB hydrogels (FIG. 19), they exhibited the least sprouting of all groups, suggesting that matrix stiffness was not the only deciding factor in the extent of vascular sprouting. This difference may be attributed to other factors, including swelling and viscoelasticity, which could influence the degree of integrin clustering and subsequent angiogenesis. To this end, we assessed the expression of integrin β1 and found lower signals in physically crosslinked dSIS hydrogels (FIG. 20), suggesting a role of moderate matrix stiffness on integrin β1 activation. A cross-sectional view of a confocal image on day 3 of a HUVEC spheroid within the 0.8 wt % dSIS-NB hydrogel (crosslinked by 0.4 wt % PEG4SH) revealed a monolayer of endothelial cells on the outermost layer of the spheroid (FIG. 21). This endothelial cell behavior was in agreement with prior studies by Duong et al. and Onoe et al. using Type I collagen and fibrin gels. The HUVEC spheroids were also manipulated into a Y-shaped pattern and embedded them within 0.8 wt % dSIS-NB hydrogels (crosslinked by 0.4 wt % PEG4SH). After two days of culture, the HUVEC spheroids fused together to create larger clusters (FIG. 22). This concept could be further applied to 3D bioprinting using cell spheroids to construct vascularized volumetric tissues in dSIS-NB hydrogels. The luminal structure of the micro neo-vasculature sprouting from the HUVEC spheroid was also visualized (FIG. 23), suggesting the potential formation of a perfusable vascular network. Current results clearly demonstrated the outstanding angiogenic properties of thiol-norbornene crosslinked dSIS-NB hydrogels over physically crosslinked dSIS gels.

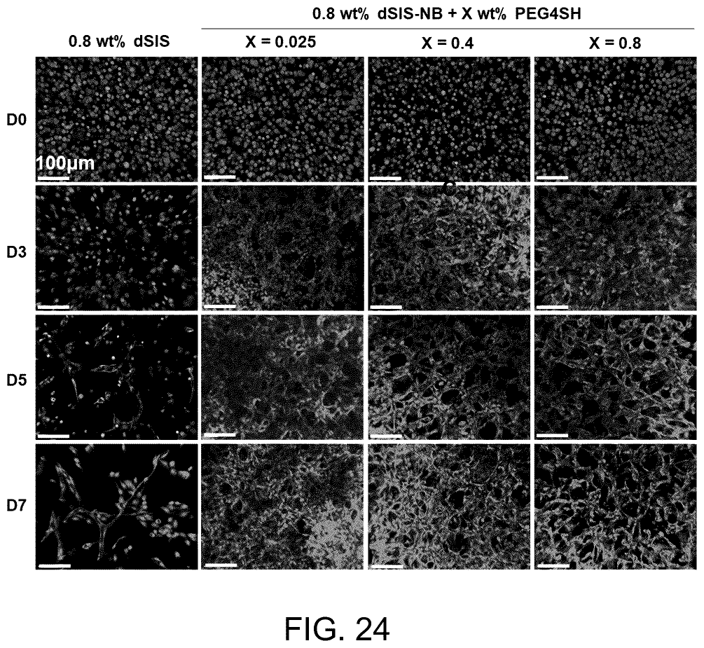

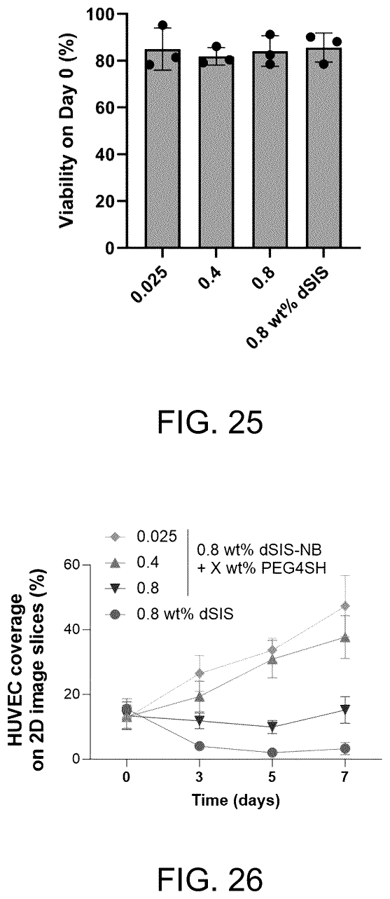

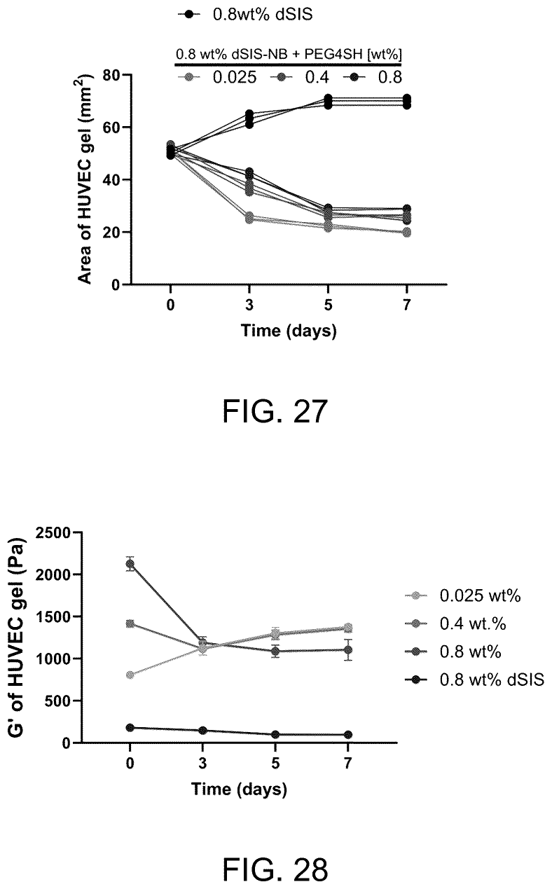

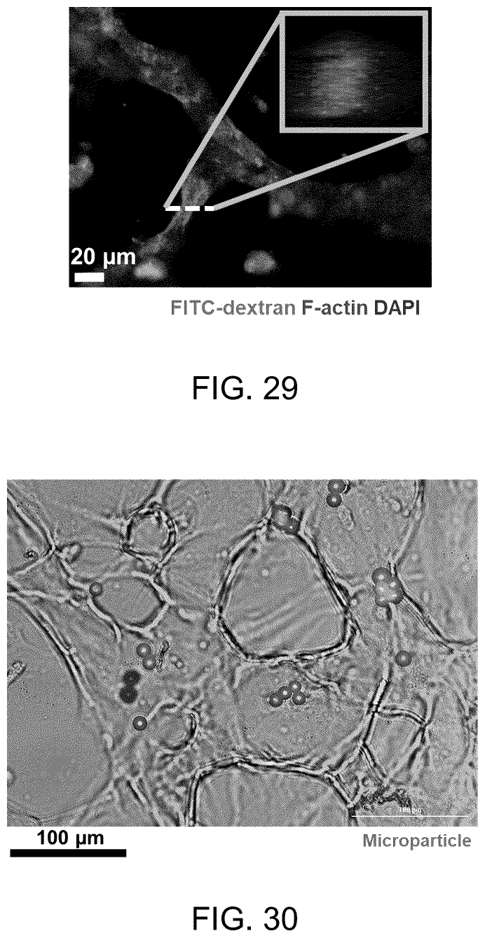

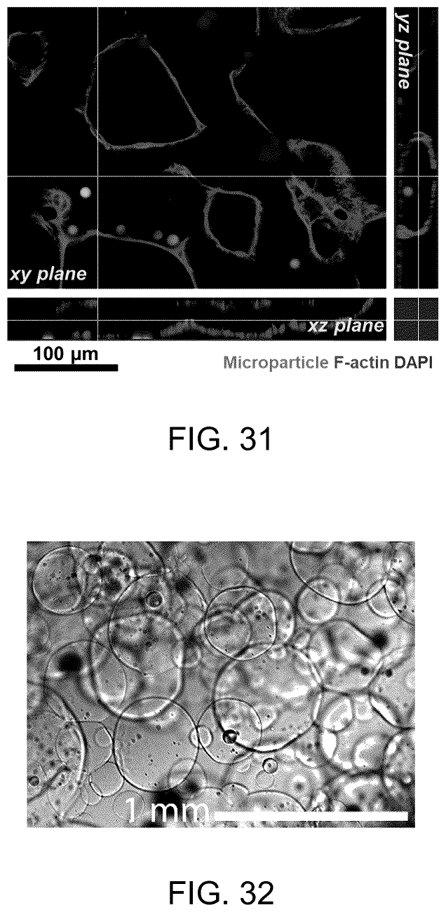

To demonstrate the utility of the new thiol-norbornene dSIS-NB hydrogels in regenerative medicine applications, human umbilical vein endothelial cells (HUVECs) were encapsulated and functional vascular network formation was evaluated. Physically gelled dSIS hydrogels were used as a control. High initial cell viability (>85%) was noted in all physical and chemically crosslinked dSIS-based hydrogels (FIG. 24, FIG. 25) and most cells remained viable in all gels throughout the 7-day culture (FIG. 24). Interestingly, cells encapsulated in the thiol-norbornene dSIS-NB hydrogels exhibited faster proliferation and more extensive network formation than the physically crosslinked dSIS hydrogels (FIG. 24). From single plane confocal images, the coverage of HUVECs as a means of evaluating vascular network formation was assessed. Cell coverage in the dSIS gel dropped from approximately 15% to 4% in 3 days, indicating a rapid regression of the newly formed vasculature. In contrast, cell coverage increased significantly over 7 days in dSIS-NB hydrogels crosslinked with 0.025 wt % and 0.4 wt % PEG4SH, but not with 0.8 wt % PEG4SH (FIG. 26). These results imply that dSIS-NB hydrogels with intermediate crosslinking density might be more advantageous for cell adhesion and migration (FIG. 9). The physically crosslinked dSIS hydrogels showed substantial swelling than the chemically crosslinked dSIS-NB hydrogels (FIG. 27), which might be detrimental in maintaining intact vascular network. On the other hand, dSIS-NB hydrogels with high crosslinking density may hinder cell migration owing to the denser network structure. As hydrogel stiffness appeared to be a critical factor in cell fate processes, the shear moduli of cell-laden hydrogels was characterized over 7 days. As shown in FIG. 28, physically crosslinked dSIS hydrogels were soft (G′<200 Pa) and fragile. Conversely, chemically crosslinked dSIS-NB hydrogels were considerably stiffer, with G′ ranging from ˜750 Pa to 1,500 Pa. The stiffness of dSIS-NB gels crosslinked by 0.4 wt % and 0.8 wt % PEG4SH dropped markedly during the first three days and stabilized at around 1,200 Pa afterward. This result suggests that intercellular network and cell-matrix interactions could contribute to the reinforcement of dSIS-NB gels (FIG. 7 and FIG. 28). Most biologically derived hydrogels, such as Matrigel, dSIS, dSIS-MA, gelatin methacryloyl (GelMA), and hyaluronic acid methacrylate (HAMA), typically exhibited excessive swelling during culture. In contrast, the cell-encapsulated dSIS-NB hydrogels maintained their sizes and mechanical properties (after 3 days of culture) owing to the chemical thiol-norbornene crosslinks and cell-material interactions (FIG. 27 and FIG. 28). To show the functionality of the vasculature formed within dSIS-NB hydrogels, on day 5, a vascularized hydrogel (0.8 wt dSIS-NB, 0.8 wt PEG4SH) was incubated in cell culture media containing 0.5 mg/mL 40 kDa FITC-Dextran for 16 h then fixed, and immunostained. FITC-Dextran fully occupied the lumen structure of the HUVEC channel (FIG. 29). A 3D vascular network was also formed in the middle channel of a u-side (80326, ibidi) using the same hydrogel formulation as the Dextran experiment. FIG. 30 and FIG. 31 demonstrated the lumen structure of microscopic interconnected HUVEC channels on day 3 within the hydrogel. Fluorescent microparticles were then perfused through the microvascular network (FIG. 30 and FIG. 31). The particles moved inside the vascular channels creating a visible flow. The 3D confocal cross-sectional views shown in FIG. 31 showed the microvascular network with trapped fluorescent microparticles in its lumen. The microparticle diameter was about 8 to 10 μm which was equivalent to the size of human red blood cells (7.5-8.7 μm). Meanwhile, the diameters of the vascular channels observed in FIG. 30 and FIG. 31 were approximately 10 to 110 μm. These results indicated that dSIS-NB hydrogels provided a favourable condition to quickly form a functional microvascular network in vitro without structural damage to the hydrogels.

### Example 5

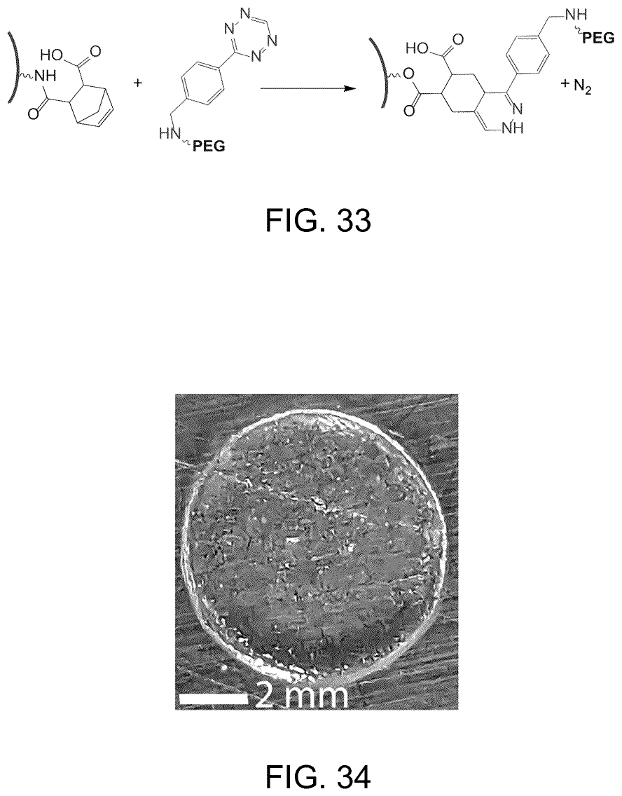

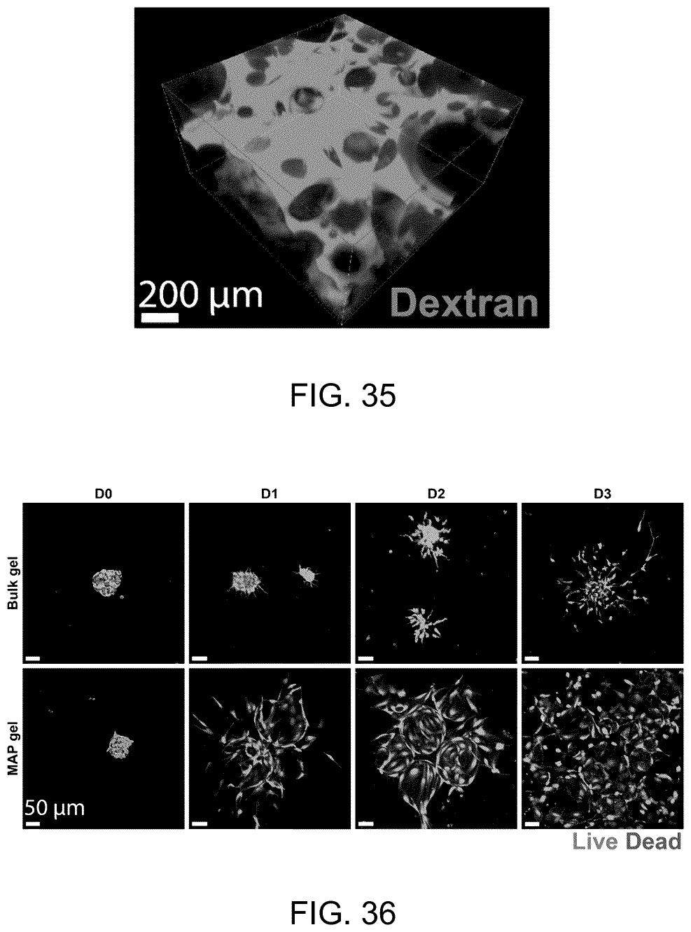

Synthesis of dSIS-NB microgels for microporous annealed particle (MAP) hydrogels. Thiol-norbornene photoclick reaction has been used to fabricate micro and nano-scale hydrogels and for annealing microgels into granular or microporous annealed particle (MAP) hydrogels. To test the feasibility of using the new dSIS-NB to formulate microgels via thiol-norbornene photo-crosslinking, a water-in-oil emulsion of 2 wt % dSIS-NB, 0.65 wt % PEG4SH, and 5 mM LAP was created. Of note, the stoichiometric ratio of thiol to norbornene (R_{[SH/NB]}) was set at 0.5, ensuring an excess of norbornene for post-gelation particulate annealing reaction. The aqueous macromer solution was emulsified in mineral oil via rigorous vortexing, followed by 365 nm light exposure for 2 minutes to produce microgels. As shown in FIG. 32, dSIS-NB microgels were successfully fabricated, with an average diameter of 212±38 μm. To create MAP gels, microgels were packed and annealed using 0.5 wt % 4-arm PEG-tetrazine (PEGTz or PEG4Tz) via inverse electron demand Diels-Alder (iEDDA) click chemistry (FIG. 33). The infused PEGTz readily reacted with excess norbornene groups on the microgel surface, creating granular MAP gels (FIG. 34). To visualize the microporosity of the MAP gels, the MAP gel was incubated with high molecular weight fluorescein isothiocyanate-labeled dextran (FITC-dextran, 2 MDa @ 1 mg/mL). FIG. 35 illustrates that FITC-dextran successfully permeated the void space in the MAP gel. The void fraction of the MAP gel was measured using ImageJ at approximately 20% while the stiffness of the MAP gel was about 670 Pa (sample size=4). Of note, the sizes of the emulsified microgels exhibited a significant degree of variation, with diameters spanning a range from 10 μm to 500 μm. Muir et al. demonstrated that higher coefficient variation in microgel size significantly reduced the extrusion force with an 18-Gauge needle for injectability of thiol-norbornene MAP hydrogels. In order to obtain microgels with more homogeneous sizes, microfluidic droplet generators should be applied to fabricate microgels.

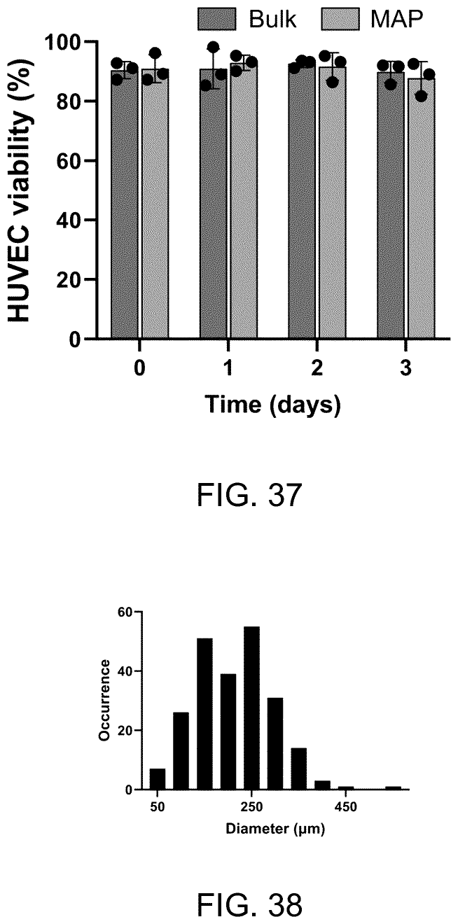

Next, HUVEC spheroids were encapsulated in dSIS-NB-based bulk gels and MAP gels to observe the effect of hydrogel microstructure on vascular cell sprouting. Live/dead staining results showed similarly high viability (>85%) of HUVECs encapsulated in both bulk and MAP gels (FIG. 36 and FIG. 37), indicative of the high cytocompatibility of the thiol-norbornene photopolymerization (for bulk cell encapsulation) and norbornene-tetrazine click reaction (for annealing microgels). The average diameter of the spheroids was similar to the average size of the microgels (c.a. 212 μm) (FIG. 32, FIG. 36, and FIG. 38). As shown in FIG. 36, HUVEC spheroids encapsulated in bulk dSIS-NB gels exhibited sprouting akin to that shown in FIGS. 16, 17, and 20-23. In contrast, cells from HUVEC spheroids encapsulated in the dSIS-NB MAP gels spread out and covered the surface of the microgels and reached confluence on days 2 and 3 (FIG. 36). Due to the presence of void compartments within MAP gels, the growth behavior of HUVECs in MAP gels closely resembles that in a 2D cell culture environment, resulting in significantly enhanced cell migration and confluence rates compared to that in bulk gels. This represents a pivotal advantage of dSIS-NB based MAP hydrogels, rendering them exceptionally well-suited for promoting angiogenesis and facilitating the formation of microvessels in tissue engineering applications especially for in vivo conditions.

Recently, Lin et al. used dECM from porcine sciatic nerve tissues to form microgels with microfluidic chips through physical crosslinking at 37° C. The microgels were enzymatically degradable for improving wound healing and tissue repair. However, the time needed for physical crosslinking of these microgels was long, around 15 to 45 min. During this long gelation time, it might be challenging to separate the microgels from coalescing. This significantly hinders the scalability of microgel production. In contrast, the gel point for thiol-norbornene photo-click reaction between dSIS-NB and PEG4SH was under 10 seconds (FIG. 3), permitting rapid production of dSIS-based microgels. Moreover, the excess norbornene moieties remaining on the surface of microgels were leveraged for tetrazine-norbornene click chemistry that annealed the microgels to be MAP gel with high void fraction (FIG. 33, FIG. 34, and FIG. 35). The high void fraction and bioactive compositions of dSIS-NB MAP gels promoted HUVEC spreading and coverage of the microgel surface quickly (FIG. 36). Of note, the gradual degradation of dSIS-NB hydrogels (FIG. 7) negates the need of scaffold removal upon the completion of desired regeneration.

### Example 6

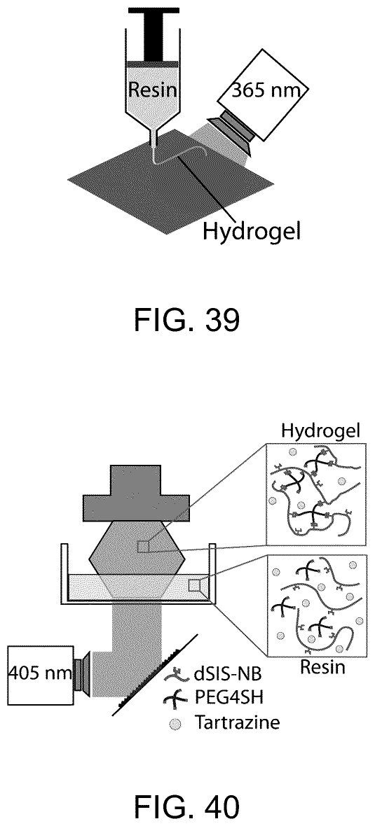

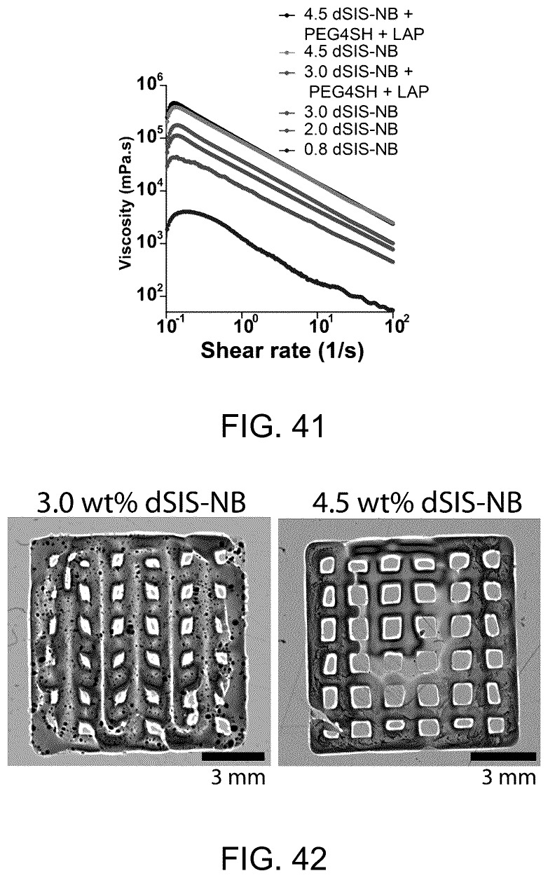

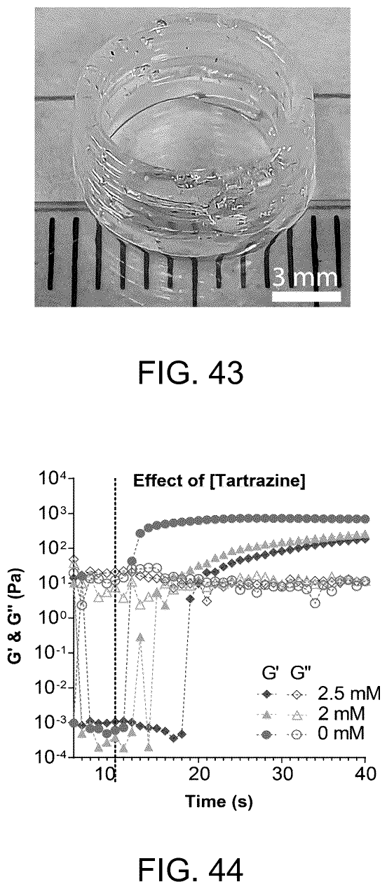

dSIS-NB as a photocrosslinkable bioink. dSIS-based gels possess collagenous structures with high aspect ratios and shear-thinning properties, which are ideal for extrusion-based bioprinting. To demonstrate the versatility of dSIS-NB as a bioink, its printability with extrusion-based (FIG. 39) and DLP (FIG. 40) bioprinters was tested. The viscosity of all dSIS-NB samples decreased as the applied shear rate increased and the addition of PEG4SH slightly increased the overall viscosity of the mixture compared to pure dSIS-NB (FIG. 41). This viscosity increase could be attributed to the highly hydrophilic macromer structure of PEG4SH. Compared to other reported studies, the viscosity of dSIS-NB at concentrations over 3 wt % was suitable for extrusion-based bioprinting. This indicates that dSIS-NB possesses adequate viscosity for bioprinting applications at higher concentrations. Indeed, we employed dSIS-NB at concentrations of 3 and 4.5 wt % (with 1 wt % PEG4SH, 5 mM LAP) as bioinks for the 3D extrusion printing. As shown in FIG. 42, both bioinks exhibited high printability, resulting in the formation of 3D square grids. Notably, the bioink containing 4.5 wt % dSIS-NB yielded higher resolution and more consistently uniform printing lines. In particular, the highly efficient thiol-norbornene photocrosslinking of dSIS-NB hydrogels enabled 3D bioprinting layer by layer. FIG. 43 shows a tube-shaped printed object with 4.5 wt % dSIS-NB with 8-mm inner diameter and 0.8-mm wall thickness. A high concentration of dSIS-NB not only enhanced printing resolution and fidelity but also provided higher bioactive contents to promote cell-matrix interactions and. Unlike the printing of other ECM-based bioinks that often requires higher temperature or non-physiological pH, the printing of dSIS-NB could be readily performed at room temperature (˜25° C.) and physiological pH. These findings suggested that dSIS-NB is a viable and appropriate selection as a bioactive bioink for extrusion-based bioprinting.

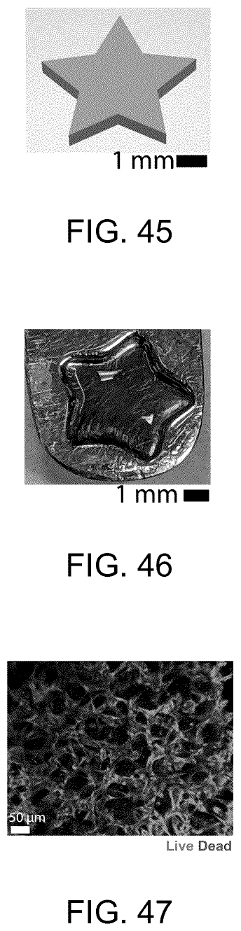

To demonstrate the utility of dSIS-NB in DLP bioprinting, a low concentration (0.8 wt %) of dSIS-NB was employed for its low viscosity, which is favorable in DLP bioprinting. Tartrazine was added as a photoabsorber, which reduced off-focal-plane polymerization. Here, we examined the influence of tartrazine on the cross-linking kinetics of LAP-initiated dSIS-NB hydrogels. In the absence of tartrazine (0 mm), the gel point (denoted as the G′/G″ cross-over time) was only 2 seconds of visible light exposure (405 nm light at 28.8 mW/cm²). The storage modulus subsequently reached a plateau (G′˜1,000 Pa) after about 8 seconds of light irradiation (FIG. 44). When tartrazine was introduced at concentrations of 2 and 2.5 mM, the gel points increased to 6 and 10 seconds, while the plateau moduli lowered to 120 Pa and 100 Pa, respectively. Further increasing tartrazine concentration could substantially delay or even entirely prevent gelation. Consequently, we selected 2 mM tartrazine as the optimal concentration for our bioink, as it provided the desired balance between enhanced DLP printing resolution and efficient gelation. FIG. 45 and FIG. 46 exhibited a computer-aided (CAD) design and a DLP printed star-shaped hydrogel, respectively. Furthermore, we encapsulated HUVECs at a density of 1.2×10⁶ cells/mL. FIG. 47 presented a live/dead 3D confocal image, illustrating the development of a continuously interconnected microvascular network within the printed hydrogel on day 3. This vascularization was fast as compared to other reported studies. Indeed, Elomaa et al. encapsulated HUVECs within 0.25 wt % rat tail collagen gel at high cell density (2×10⁶ cells/mL) and it took 7 to 14 days to form a continuously interconnected vascular network. In another example using 3 wt % GelMA hydrogels, HUVECs encapsulated at a density of 5×10⁶ cells/mL did not form interconnected vascular network until after 7 days. These results indicated that dSIS-NB could be a more efficient bioink for light-based bioprinting.

### REFERENCES

Each of the following documents is hereby expressly incorporated by reference herein in its entirety.
Y. S. Kim, M. Majid, A. J. Melchiorri, A. G. Mikos, Bioengineering & Translational Medicine 2019, 4, 83.
D. Bejleri, M. E. Davis, Advanced Healthcare Materials 2019, 8, 1801217.
D. Van Thuy, D. Thao Thi, L. Van Phu, L. Thi Huong, N. Chanh Trung, P. Huu Lam, S. Jongmo, B. Sung Hoon, K. Kyo-in, bioRxiv 2023, 2023.09.26.559212.
L. Elomaa, L. Gerbeth, A. Almalla, N. Fribiczer, A. Daneshgar, P. Tang, K. Hillebrandt, S. Seiffert, I. M. Sauer, B. Siegmund, M. Weinhart, Additive Manufacturing 2023, 64, 103439.
T. Palmosi, A. M. Tolomeo, C. Cirillo, D. Sandrin, M. Sciro, S. Negrisolo, M. Todesco, F. Caicci, M. Santoro, E. Dal Lago, M. Marchesan, M. Modesti, A. Bagno, F. Romanato, P. Grumati, A. Fabozzo, G. Gerosa, Frontiers in Bioengineering and Biotechnology 2022, 10.
U.S. F.a.D. Administration, Vol. 2023.
G. G. Giobbe, C. Crowley, C. Luni, S. Campinoti, M. Khedr, K. Kretzschmar, M. M. De Santis, E. Zambaiti, F. Michielin, L. Meran, Q. Hu, G. van Son, L. Urbani, A. Manfredi, M. Giomo, S. Eaton, D. Cacchiarelli, V. S. W. Li, H. Clevers, P. Bonfanti, N. Elvassore, P. De Coppi, Nature Communications 2019, 10, 5658.
H.-K. Lin, S. Y. Godiwalla, B. Palmer, D. Frimberger, Q. Yang, S. V. Madihally, K.-M. Fung, B. P. Kropp, Tissue Engineering Part B: Reviews 2013, 20, 73.
Y. Zhang, S. Shi, Q. Xu, Q. Zhang, R. M. Shanti, A. D. Le, Journal of Dental Research 2018, 98, 225.
G. Cao, Y. Huang, K. Li, Y. Fan, H. Xie, X. Li, Journal of Materials Chemistry B 2019, 7, 5038.
S. Yeleswarapu, A. Dash, S. Chameettachal, F. Pati, Biomaterials Advances 2023, 152, 213494.
W. Wang, X. Zhang, N.-N. Chao, T.-W. Qin, W. Ding, Y. Zhang, J.-W. Sang, J.-C. Luo, Acta Biomaterialia 2016, 29, 135.
L. T. Saldin, M. C. Cramer, S. S. Velankar, L. J. White, S. F. Badylak, Acta Biomaterialia 2017, 49, 1.
T. T. T. Nguyen, V. T. Duong, L. T. Huong, K. I. Koo, presented at 2022 International Workshop on Intelligent Systems (IWIS), 17-19 Aug. 2022, 2022.
D. F. Williams, Frontiers in Bioengineering and Biotechnology 2019, 7.
M. Brown, J. Li, C. Moraes, M. Tabrizian, N. Y. K. Li-Jessen, Biomaterials 2022, 289, 121786.
A. G. P. de Paula, J. D. de Lima, T. S. B. Bastos, A. P. Czaikovski, R. B. dos Santos Luz, B. S. Yuasa, C. C. S. Smanioto, A. W. Robert, T. T. Braga, ACS Omega 2023, 8, 22256.
V. T. Duong, T. T. Dang, C. H. Hwang, S. H. Back, K.-i. Koo, Biofabrication 2020, 12, 045033.
J. Ahn, T. Sen, D. Lee, H. Kim, J. Y. Lee, H. S. Koo, J. Y. Kim, J. Kim, J. Jang, Y.-J. Kang, D.-W. Cho, Advanced Functional Materials 2023, 33, 2214291.
R. S. Hewawasam, R. Blomberg, P. Šerbedžija, C. M. Magin, ACS Applied Materials & Interfaces 2023, 15, 15071.
C.-C. Lin, A. Raza, H. Shih, Biomaterials 2011, 32, 9685.
A. Phaniendra, D. B. Jestadi, L. Periyasamy, Indian Journal of Clinical Biochemistry 2015, 30, 11.
V. T. Duong, C.-C. Lin, Macromolecular Bioscience 2023, n/a, 2300213.
K. Vats, G. Marsh, K. Harding, I. Zampetakis, R. E. Waugh, D. S. W. Benoit, Journal of Biomedical Materials Research Part A 2017, 105, 1112.
M. R. Arkenberg, H. D. Nguyen, C.-C. Lin, Journal of Materials Chemistry B 2020, 8, 7835.
M. W. Tibbitt, A. M. Kloxin, L. A. Sawicki, K. S. Anseth, Macromolecules 2013, 46, 2785.
M. H. Kim, C.-C. Lin, ACS Applied Materials & Interfaces 2023.
P. N. Bernal, P. Delrot, D. Loterie, Y. Li, J. Malda, C. Moser, R. Levato, Advanced Materials 2019, 31, 1904209.
M. H. Kim, C.-C. Lin, Biofabrication 2021, 13, 045023.
R. Rizzo, D. Ruetsche, H. Liu, M. Zenobi-Wong, Advanced Materials 2021, 33, 2102900.
S. Das, S.-W. Kim, Y.-J. Choi, S. Lee, S.-H. Lee, J.-S. Kong, H.-J. Park, D.-W. Cho, J. Jang, Acta Biomaterialia 2019, 95, 188.
T. H. Petersen, E. A. Calle, M. B. Colehour, L. E. Niklason, Cells Tissues Organs 2011, 195, 222.
T. Bongolan, J. Whiteley, J. Castillo-Prado, A. Fantin, B. Larsen, C. J. Wong, L. Mazilescu, M. Kawamura, P. Urbanellis, A. Jonebring, E. Salter, G. Collingridge, R. Gladdy, R. Hicks, A.-C. Gingras, M. Selzner, I. M. Rogers, Biomaterials Science 2022, 10, 2972.
Y. Chen, S. Geerts, M. Jaramillo, B. E. Uygun, in Decellularized Scaffolds and Organogenesis: Methods and Protocols, (Ed: K. Turksen), Springer New York, New York, NY 2018.
H. Kim, J.-H. Jang, W. Han, H.-J. Hwang, J. Jang, J. Y. Kim, D.-W. Cho, Biomaterials 2023, 292, 121941.
D. M. Faulk, C. A. Carruthers, H. J. Warner, C. R. Kramer, J. E. Reing, L. Zhang, A. D'Amore, S. F. Badylak, Acta Biomaterialia 2014, 10, 183.
O. Syed, N. J. Walters, R. M. Day, H.-W. Kim, J. C. Knowles, Acta Biomaterialia 2014, 10, 5043.
Z. Mũnoz, H. Shih, C.-C. Lin, Biomaterials Science 2014, 2, 1063.
M. H. Kim, H. Nguyen, C.-Y. Chang, C.-C. Lin, ACS Biomaterials Science & Engineering 2021, 7, 4196.
F.-Y. Lin, C.-C. Lin, ACS Macro Letters 2021, 10, 341.
C.-C. Lin, C. S. Ki, H. Shih, Journal of Applied Polymer Science 2015, 132.
H. Onoe, T. Okitsu, A. Itou, M. Kato-Negishi, R. Gojo, D. Kiriya, K. Sato, S. Miura, S. Iwanaga, K. Kuribayashi-Shigetomi, Y. T. Matsunaga, Y. Shimoyama, S. Takeuchi, Nature Materials 2013, 12, 584.
Y. Jun, E. Kang, S. Chae, S.-H. Lee, Lab on a Chip 2014, 14, 2145.
G. T. Fallenstein, V. D. Hulce, J. W. Melvin, Journal of Biomechanics 1969, 2, 217.
L. S. Glaus, L. Hofstetter, A. Guekos, P. Schweinhardt, J. Swanenburg, European Journal of Applied Physiology 2021, 121, 2277.
T. Luo, B. Tan, L. Zhu, Y. Wang, J. Liao, Frontiers in Bioengineering and Biotechnology 2022, 10.
D. A. D. Parry, G. R. G. Barnes, A. S. Craig, D. C. Phillips, Proceedings of the Royal Society of London. Series B. Biological Sciences 1997, 203, 305.
T. Ushiki, Kaibogaku zasshi. Journal of anatomy 1992, 67, 186.
A. Sorushanova, L. M. Delgado, Z. Wu, N. Shologu, A. Kshirsagar, R. Raghunath, A. M. Mullen, Y. Bayon, A. Pandit, M. Raghunath, D. I. Zeugolis, Advanced Materials 2019, 31, 1801651.
K. M. Yamada, A. D. Doyle, J. Lu, Trends in Cell Biology 2022, 32, 883.
S. R. Caliari, J. A. Burdick, Nature Methods 2016, 13, 405.
N. T. Lam, H. Lam, N. M. Sturdivant, K. Balachandran, Biomedical Materials 2017, 12, 045013.
Z. Sun, H. Xiong, T. Lou, W. Liu, Y. Xu, S. Yu, H. Wang, W. Liu, L. Yang, C. Zhou, C. Fan, Gels (Basel, Switzerland) 2023, 9.
M. Zhu, Y. Wang, G. Ferracci, J. Zheng, N.-J. Cho, B. H. Lee, Scientific Reports 2019, 9, 6863.
G. Camci-Unal, D. Cuttica, N. Annabi, D. Demarchi, A. Khademhosseini, Biomacromolecules 2013, 14, 1085.
M. Diez-Silva, M. Dao, J. Han, C.-T. Lim, S. Suresh, MRS Bulletin 2010, 35, 382.
H. T. Le, H. L. Phan, A. Lenshof, V. T. Duong, C. Choi, C. Cha, T. Laurell, K.-i. Koo, Biofabrication 2024, 16, 015009.
E. Martina, M. Degen, C. Ruegg, A. Merlo, M. M. Lino, R. Chiquet-Ehrismann, F. Brellier, The FASEB Journal 2010, 24, 778.
F. Tetzlaff, A. Fischer, Bio-protocol 2018, 8, e2995.
E. Mehes, M. Barath, M. Gulyas, E. Bugyik, M. Geiszt, A. Szoor, A. Lanyi, A. Czirok, Scientific Reports 2019, 9, 14363.
O. J. Mezu-Ndubuisi, A. Maheshwari, Pediatric Research 2021, 89, 1619.
D. Banerjee, Y. P. Singh, P. Datta, V. Ozbolat, A. O'Donnell, M. Yeo, I. T. Ozbolat, Biomaterials 2022, 291, 121881.
P. M. Kharkar, M. S. Rehmann, K. M. Skeens, E. Maverakis, A. M. Kloxin, ACS Biomaterials Science & Engineering 2016, 2, 165.
N. J. Darling, W. Xi, E. Sideris, A. R. Anderson, C. Pong, S. T. Carmichael, T. Segura, Advanced Healthcare Materials 2020, 9, 1901391.
N. H. Dimmitt, M. R. Arkenberg, M. M. de Lima Perini, J. Li, C.-C. Lin, ACS Biomaterials Science & Engineering 2022, 8, 4262.
F. Reed George, F. Lynn, D. Meade Bruce, Clinical and Vaccine Immunology 2002, 9, 1235.
V. G. Muir, T. H. Qazi, J. Shan, J. Groll, J. A. Burdick, ACS Biomaterials Science & Engineering 2021, 7, 4269.
Z. Jiang, F.-Y. Lin, K. Jiang, H. Nguyen, C.-Y. Chang, C.-C. Lin, Biomaterials Advances 2022, 134, 112712.
D. R. Griffin, W. M. Weaver, P. O. Scumpia, D. Di Carlo, T. Segura, Nature Materials 2015, 14, 737.
Z. Lin, Z. Rao, J. Chen, H. Chu, J. Zhou, L. Yang, D. Quan, Y. Bai, ACS Biomaterials Science & Engineering 2022, 8, 1644.
V. Mittal, Annual Review of Pathology: Mechanisms of Disease 2018, 13, 395.
D. Ribatti, R. Tamma, T. Annese, Translational oncology 2020, 13, 100773.
J. I. Herschkowitz, K. Simin, V. J. Weigman, I. Mikaelian, J. Usary, Z. Hu, K. E. Rasmussen, L. P. Jones, S. Assefnia, S. Chandrasekharan, M. G. Backlund, Y. Yin, A. I. Khramtsov, R. Bastein, J. Quackenbush, R. I. Glazer, P. H. Brown, J. E. Green, L. Kopelovich, P. A. Furth, J. P. Palazzo, O. I. Olopade, P. S. Bernard, G. A. Churchill, T. Van Dyke, C. M. Perou, Genome Biology 2007, 8, R76.
J. M. Lee, S. Dedhar, R. Kalluri, E. W. Thompson, Journal of Cell Biology 2006, 172, 973.
M. J. Buehler, Proceedings of the National Academy of Sciences 2006, 103, 12285.
S. M. Siadat, A. A. Silverman, C. A. DiMarzio, J. W. Ruberti, Journal of Structural Biology 2021, 213, 107697.
Y. Li, C. Qiao, L. Shi, Q. Jiang, T. Li, Journal of Macromolecular Science, Part B 2014, 53, 893.
V. T. Duong, Jong Pal Kim, Kwangsoo Kim, Hyoungho Ko, Chang Ho Hwang, Kyo-in Koo, Journal of Biomedical Engineering Research 2018, 39, 188.
P. A. Amorim, M. A. d'Ávila, R. Anand, P. Moldenaers, P. Van Puyvelde, V. Bloemen, Bioprinting 2021, 22, e00129.
Y. Chen, X. Xiong, X. Liu, R. Cui, C. Wang, G. Zhao, W. Zhi, M. Lu, K. Duan, J. Weng, S. Qu, J. Ge, Journal of Materials Chemistry B 2020, 8, 5500.
S. A. Wilson, L. M. Cross, C. W. Peak, A. K. Gaharwar, ACS Applied Materials & Interfaces 2017, 9, 43449.
A. Abaci, M. Guvendiren, Advanced Healthcare Materials 2020, 9, 2000734.
K. K. Moncal, V. Ozbolat, P. Datta, D. N. Heo, I. T. Ozbolat, Journal of Materials Science: Materials in Medicine 2019, 30, 55.
J. Huh, Y.-W. Moon, J. Park, A. Atala, J. J. Yoo, S. J. Lee, Biofabrication 2021, 13, 034103.
L. Elomaa, M. Lindner, R. Leben, R. Niesner, M. Weinhart, Biofabrication 2023, 15, 015004.
Y. Zuo, X. Liu, D. Wei, J. Sun, W. Xiao, H. Zhao, L. Guo, Q. Wei, H. Fan, X. Zhang, ACS Applied Materials & Interfaces 2015, 7, 10386.
J. Kleeff, K. Fukahi, M. E. Lopez, H. Friess, M. W. Büchler, B. A. Sosnowski, M. Korc, Cancer Gene Therapy 2002, 9, 522.
K. E. Richards, A. E. Zeleniak, M. L. Fishel, J. Wu, L. E. Littlepage, R. Hill, Oncogene 2017, 36, 1770.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising decellularized extracellular matrices, wherein the decellularized extracellular matrices comprise one or more norbornene substituents.

2. The composition of claim 1, wherein the one or more norbornene substituent is attached to one or more primary amines of the decellularized extracellular matrices.

3. The composition of claim 1, wherein the decellularized extracellular matrices are derived from a mammal.

4. A macromer for producing a hydrogel, the macromer comprising decellularized extracellular matrices, wherein the decellularized extracellular matrices comprise one or more norbornene substituents.

5. A method for making a photocrosslinkable macromer, the method comprising:
reacting decellularized extracellular matrices (dECM) with carbic anhydride and a nucleophilic catalyst to yield a norbornene-functionalized dECM.

6. The method of claim 5, wherein the dECM comprises one or more primary amines.

7. The method of claim 6, wherein the photocrosslinkable macromer comprises one or more amide linkages formed by the one or amines of the dECM and a carboxyl domain of the norbornene.

8. The method of claim 5, wherein the nucleophilic catalyst comprises triethylamine.

9. A method for making a hydrogel, comprising:
reacting decellularized extracellular matrices (dECM) with carbic anhydride and a nucleophilic catalyst to yield a norbornene-functionalized dECM; and
reacting the norbornene-functionalized dECM with at least one thiol-bearing compound.

10. The method of claim 9 wherein the reacting the norbornene-functionalized dECM with at least one thiol-bearing compound comprises exposing the norbornene-functionalized dECM and the thiol-bearing compound to electromagnetic radiation having a wavelength from 365 nm to 405 nm.

11. The method of claim 9, wherein the thiol-bearing compound is selected from the group consisting of 1,4-dithiothreitol, 4-arm thiolated PEG, a peptide that includes more than one cysteine residue, a natural or thiolated protein, thiolated gelatin, thiolated hyaluronic acid, thiolated collagens, and combinations thereof.

12. The method of claim 9, wherein the thiol-bearing compound comprises dithiothreitol.

13. The method of claim 9, wherein the hydrogel comprises an orthogonal thiol-norbornene hydrogel.

14. A hydrogel produced according to the method of claim 9.

15. Use of a hydrogel in accordance with claim 9 for a purpose selected from the group consisting of as a substrate for short-term 2D cell culturing, 3D in situ encapsulation of viable cells, controlled delivery of drugs and other bioactive materials, tissue engineering, tissue regeneration, creating micropores in a bulk polyethylene glycol-based hydrogel, creating a sacrificial material in a 3D bioprinted article, creating a sacrificial material in a photolithography-based biofabrication product, and creating a sacrificial bioink for extrusion-based or photolithography-based 3D bioproducts.


SEQUENCE LISTING (1 sequence)

SEQ ID NO: 1 - protein, 17 aa, synthetic construct
KLTWQELYQLKYKGIGC